(19) United States
(12) Patent Application Publication
(10) Pub. No.: US 2024/0312584 A1
(43) Pub. Date: Sep. 19, 2024

(54) LIFESTYLE IMPROVEMENT SYSTEM, PORTABLE TERMINAL, AND CONTROL METHOD

(71) Applicant: Panasonic Intellectual Property Management Co., Ltd., Osaka (JP)

(72) Inventors: Masafumi SATO, Osaka (JP); Tomoki OGAWA, Osaka (JP); Tomonori NAKAMURA, Osaka (JP); Kohsuke YAMAMOTO, Osaka (JP)

(21) Appl. No.: 18/572,085

(22) PCT Filed: Jun. 30, 2022
(86) PCT No.: PCT/JP2022/026335

§ 371 Date: Dec. 19, 2023

Related U.S. Application Data
(60) Provisional application No. 63/216,859, filed on Jun. 30, 2021.

(51) Int. Cl. G16H 20/00; A61B 5/00; A61B 5/021; A61B 5/1171; A61B 5/1172; G16H 10/60

(52) U.S. Cl. CPC G16H 20/00; A61B 5/0002; A61B 5/021; A61B 5/1172; A61B 5/1176; A61B 5/4809; A61B 5/7435; G16H 10/60; A61B 2560/0242; A61B 2560/0431; A61B 2560/0462; A61B 2562/0204; A61B 2562/0219; A61B 2562/0223

(57) ABSTRACT

A lifestyle improvement system includes: an obtainer that obtains bed-time information about an act of going to bed by the user and history information indicating a history of blood pressure measurement by the user; a determiner that determines whether the user has taken a blood pressure measurement in a period of a predetermined amount of time ending when the user goes to bed, based on the bed-time information and the history information; and a notifier that notifies the user with first timing information regarding an optimal first timing for blood pressure measurement when the determiner determines that the user has not taken the blood pressure measurement in the period.

### CROSS-REFERENCE OF RELATED APPLICATIONS

This application is the U.S. National Phase under 35 U.S.C. § 371 of International Patent Application No. PCT/JP2022/026335, filed on Jun. 30, 2022, which in turn claims the benefit of U.S. Provisional Patent Application No. 63/216,859, filed on Jun. 30, 2021, the entire disclosures of which Applications are incorporated by reference herein.

### TECHNICAL FIELD

The present disclosure relates to a lifestyle improvement system, a portable terminal, and a control method.

### BACKGROUND ART

PTL 1 discloses a technique for displaying a prompt for taking a blood pressure measurement at a preset time.

### CITATION LIST

### Patent Literature

[PTL 1] Japanese Unexamined Patent Application Publication No. 2022-11919

### SUMMARY OF INVENTION

### Technical Problem

The present disclosure provides a lifestyle improvement system, etc., that can support taking blood pressure measurements at appropriate timings.

### Solution to Problem

A lifestyle improvement system according to one aspect of the present disclosure includes: an obtainer that obtains bed-time information about an act of going to bed by a user and history information indicating a history of blood pressure measurement by the user; a determiner that determines whether the user has taken a blood pressure measurement in a period of a predetermined amount of time ending when the user goes to bed, based on the bed-time information and the history information; and a notifier that notifies the user with first timing information regarding an optimal first timing for blood pressure measurement when the determiner determines that the user has not taken the blood pressure measurement in the period.

A portable terminal according to one aspect of the present disclosure is connected to a server over a network. The server includes: an obtainer that obtains bed-time information about an act of going to bed by a user and history information indicating a history of blood pressure measurement by the user; a determiner that determines whether the user has taken a blood pressure measurement in a period of a predetermined amount of time ending when the user goes to bed, based on the bed-time information and the history information; and a notifier that notifies the user with timing information regarding an optimal timing for blood pressure measurement when the determiner determines that the user has not taken the blood pressure measurement in the period. The portable terminal includes: a receiver that receives the timing information; and an informer that informs the user of an optimal timing to measure blood pressure, based on the timing information.

A control method according to one aspect of the present disclosure is a control method of a lifestyle improvement system including one or more processors and an informer, and includes: obtaining, by the one or more processors, bed-time information about an act of going to bed by a user and history information indicating a history of blood pressure measurement by the user; and informing, by the informer, the user of timing information regarding an optimal timing for blood pressure measurement when it is determined, based on the bed-time information and the history information, that the user has not taken a blood pressure measurement in a period of a predetermined amount of time ending when the user goes to bed.

These general or specific aspects may be implemented as a device/apparatus, an integrated circuit, a computer program, or a computer-readable recording medium such as a CD-ROM, or any combination thereof.

### Advantageous Effects of Invention

The lifestyle improvement system, etc., according to present disclosure can support taking blood pressure measurements at appropriate timings.

### BRIEF DESCRIPTION OF DRAWINGS

FIG. 1 is a graph for illustrating changes in disease structure.

FIG. 2 is a graph illustrating percentages of medical expenditures for different lifestyle-related diseases.

FIG. 3 is a graph illustrating the relationship between blood pressure categories and stroke incidence.

FIG. 4 illustrates factors that reduce blood pressure.

FIG. 5 illustrates factors that prevent people from continuing to improve their lifestyle habits.

FIG. 6 is a diagram illustrating a model of behavior change.

FIG. 7 illustrates an example of a case in which a plurality of blood pressure measurements were taken at predetermined time intervals during a day.

FIG. 8 illustrates one example of a functional block diagram of a lifestyle improvement system according to an embodiment.

FIG. 9 illustrates the configuration of a device when the device is a refrigerator capable of communication.

FIG. 10 illustrates the configuration of a portable terminal in detail.

FIG. 11 is a sequence diagram illustrating a first example of operations related to blood pressure measurement performed by a lifestyle improvement system.

FIG. 12 is a flowchart illustrating the processes of determining the optimal timing for blood pressure measurement and notifying the timing information that are performed in the first example.

FIG. 13 is a diagram illustrating one example of informing the user so as to prompt the user to measure blood pressure.

FIG. 14 is a flowchart illustrating another example of the processes of determining the optimal timing for blood pressure measurement and notifying the timing information.

FIG. 15 is a diagram illustrating one example of informing the user so as to prompt the user to measure blood pressure after urinating.

FIG. 16 illustrates one example of a UI that accepts input of a timing at which a blood pressure measurement was taken.

FIG. 17 is a sequence diagram illustrating a second example of operations related to blood pressure measurement performed by a lifestyle improvement system.

FIG. 18 is a flowchart illustrating the processes of determining the optimal timing for blood pressure measurement and notifying the timing information that are performed in the second example.

FIG. 19 is a sequence diagram illustrating a third example of operations related to blood pressure measurement performed by a lifestyle improvement system.

FIG. 20 is a sequence diagram illustrating a fourth example of operations related to blood pressure measurement performed by a lifestyle improvement system.

FIG. 21 is a flowchart illustrating one example of operations related to input of food consumption log entries in the lifestyle improvement system.

FIG. 22 is a flowchart illustrating a first example of a process of inputting a food consumption log.

FIG. 23 is a flowchart illustrating a second example of a process of inputting a food consumption log.

FIG. 24 is a flowchart illustrating a third example of a process of inputting a food consumption log.

FIG. 25 is a flowchart illustrating a fourth example of a process of inputting a food consumption log.

FIG. 26 is a flowchart illustrating one example of the process of displaying blood pressure measurement results and the food consumption log in association with each other.

FIG. 27 illustrates one example of a UI including one or more second food consumption logs.

### DESCRIPTION OF EMBODIMENTS

### Background

The background of the invention will be explained.

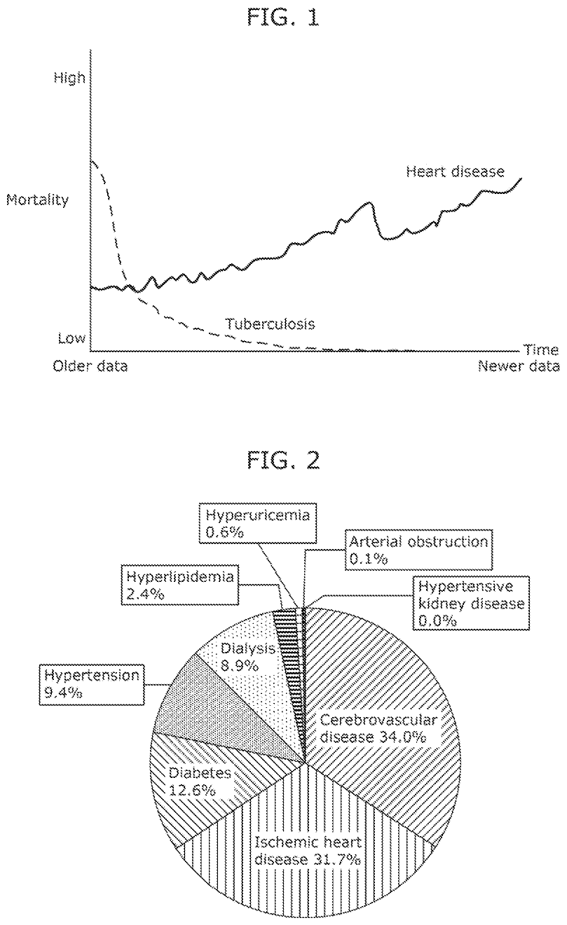

FIG. 1 is a graph for illustrating changes in disease structure.

As illustrated in FIG. 1, the structure of diseases affecting the Japanese population has shifted from infectious diseases to lifestyle-related diseases as sanitary conditions improved in the postwar period. For example, mortality from infectious diseases such as tuberculosis and pneumonia has been decreasing over time, while mortality from lifestyle-related diseases such as heart disease has been increasing. Lifestyle-related diseases are difficult to cure, so the cost of treatment is an ongoing problem. Additionally, in lifestyle-related diseases, the intensity of the patient's subjective symptoms does not correspond to the necessity of medical intervention.

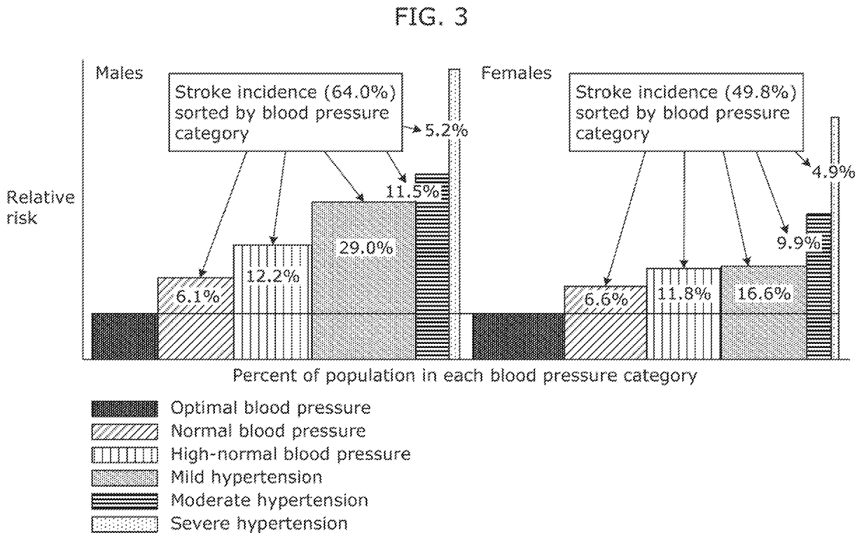

FIG. 2 is a graph illustrating percentages of medical expenditures for different lifestyle-related diseases. FIG. 3 is a graph illustrating the relationship between blood pressure categories and stroke incidence.

As illustrated in FIG. 2, about 75% of the medical expenditures for lifestyle-related diseases are spent on diseases in which high blood pressure is the main cause (cerebrovascular disease, ischemic heart disease, and hypertension). Multiple studies have also found that the risk of disease increases with higher blood pressure. For example, as illustrated in FIG. 3, the risk of stroke increases with higher blood pressure.

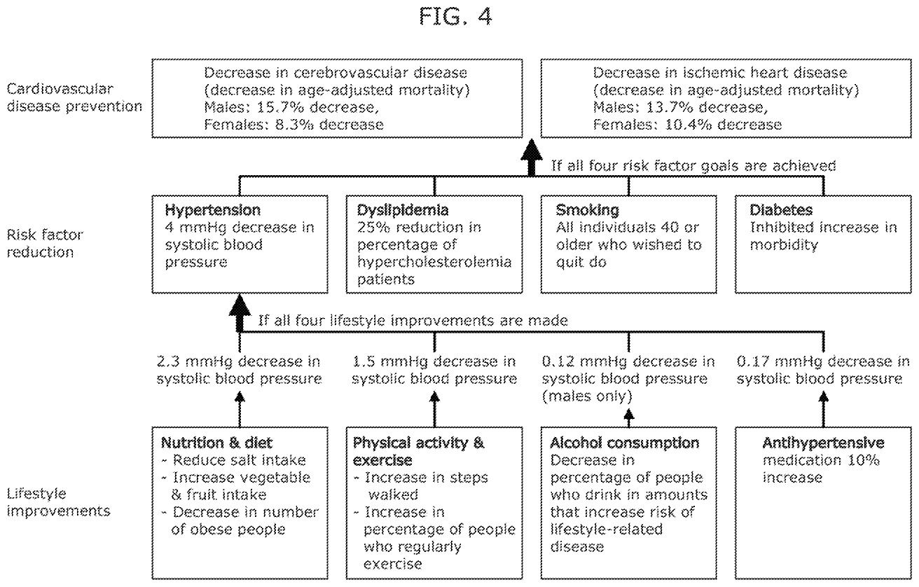

FIG. 4 illustrates factors that reduce blood pressure. FIG. 4 is a diagram based on the Guidelines for the Management of Hypertension 2019.

As illustrated in FIG. 4, four measures are considered important to reduce blood pressure: (1) nutrition and diet, (2) physical activity and exercise, (3) alcohol consumption, and (4) antihypertensive medication rates. Of these, (1) nutrition and diet, (2) physical activity and exercise, and (3) alcohol consumption are not medical measures, but measures related to improving one's everyday lifestyle. In other words, improving one's everyday lifestyle is important for reducing lifestyle-related diseases.

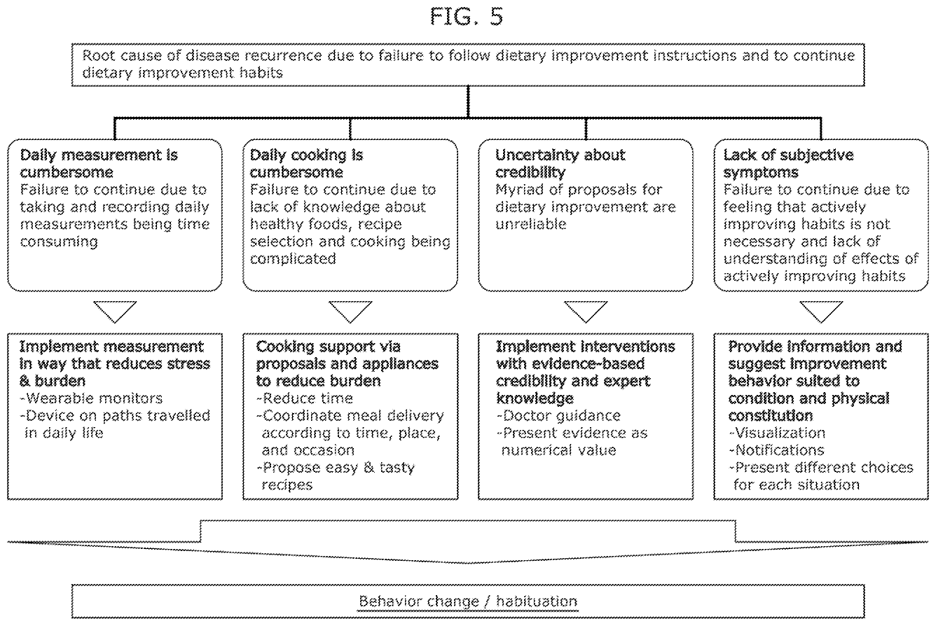

FIG. 5 illustrates factors that prevent people from continuing to improve their lifestyle habits.

In order to improve lifestyle habits in patients with lifestyle-related diseases, it is necessary to habituate behaviors that lead to good health on a patient by patient basis. If a person is able to habituate behaviors that lead to good health, it will help promote and maintain their health.

However, it is difficult to sustain lifestyle improvement behavior without following lifestyle (for example, diet) improvement instructions. Four conceivable factors for this difficulty are, for example, (1) daily measurement is cumbersome, (2) daily cooking is cumbersome, (3) uncertainty about the credibility of the information, and (4) lack of subjective symptoms. The four factors can be improved to habituate lifestyle improvement behaviors (behavior change). For example, (1) could be improved by implementing measurement in a way that reduces stress and burden. For example, (2) could be improved by implementing meal proposals that reduce the burden of cooking and implementing cooking support via home appliances. For example, (3) could be improved by implementing interventions with evidence-based credibility and expert knowledge. For example, (4) could be improved by providing information and suggesting improvement behavior suited to the condition and physical constitution of the individual.

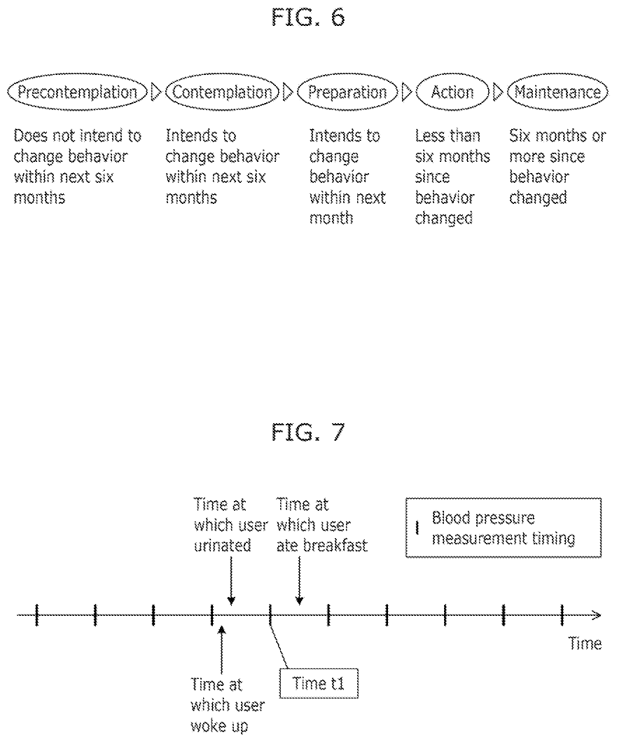

FIG. 6 is a diagram illustrating a model of behavior change.

It is common for individuals to change their behavior in gradual stages. As illustrated in FIG. 6, individuals are considered to progress through five stages of behavior change: precontemplation, contemplation, preparation, action, and maintenance. These five stages make up the model of behavior change, which is being researched and practiced on various health-related behaviors. In order to advance even one stage of behavior change, it is first necessary to know which stage the individual is in and to work with them according to the stage.

For example, the Ministry of Health, Labour and Welfare website (https://www.e-healthnet.mhlw.go.jp/information/exercise/s-07-001.html) discloses a model of behavior change and stage-appropriate approaches, which are described below.

Approaches for the precontemplation stage include, for example, raising awareness, learning about the benefits of physical activity, emotional experiences, realizing that the status quo needs to change, re-evaluating one's environment, and realizing how one's behavior impacts their surroundings.

Approaches for the contemplation stage include, for example, re-evaluation of oneself, and presenting physical inactivity as harmful and physical activity as beneficial.

Approaches for the preparation stage include, for example, personal determination and having confidence in one's ability to perform physical activity well and declaring to others that one is ready to begin physical activity.

Approaches for the action and maintenance stages include, for example, behavior replacement, replacing unhealthy behaviors with healthy ones (for example, using physical activity instead of alcohol to cope with stress), supportive relationships, using the support of others to continue physical activity, reinforcement management, giving ‘rewards’ for continuing physical activity, stimulus control, and creating an environment conducive to physical activity.

### (Smartphone Apps for Supporting Improvement Behavior)

To encourage improvement behavior, daily blood pressure measurements and food consumption management are useful for hypertensive patients. For example, a smartphone app that allows users to enter and manage their blood pressure measured on a daily basis is known. Such an app automatically obtains blood pressure measurement results from a blood pressure monitor (measurement device) connected to the smartphone via Bluetooth (registered trademark), and manages the blood pressure measurement time and blood pressure measurement results in association with each other.

For example, a smartphone apps that allows users to enter and manage their food consumption is known. Such an app uses, for example, AI to perform image recognition on an image of a meal captured using the smartphone camera to estimate the dishes of the meal, and allows the user to select dishes from among candidates listed based on the results of the estimation to manage the selected dishes.

### (Blood Pressure Monitor)

In recent years, wearable cuffless blood pressure monitors have been developed to automatically measure blood pressure at regular intervals. Cuffless blood pressure monitors are approved as medical devices in some countries or regions.

Unlike conventional blood pressure monitors, cuffless blood pressure monitors are devices that are worn, for example, on the wrist or upper arm, and enable frequent and automatic blood pressure measurements at predetermined times without the user noticing, such as at 30-minute intervals, or when predetermined conditions are met (for example, when the user is at rest and there is little movement).

Conventionally, a blood pressure monitor with a cuff called Ambulatory Blood Pressure Monitoring (ABPM) has been used to obtain frequent blood pressure readings (for example, hourly blood pressure readings), but the periodic tightening of the cuff places stress on the subject's body. For this reason, frequent blood pressure values are rarely measured except in medical facilities when truly needed.

On the other hand, rather than using a cuff, cuffless blood pressure monitors obtain blood pressure readings by estimating blood pressure based on, for example, pulse waves obtained from an optical sensor, with little or no tightening of the body measurement position (wrist, upper arm, etc.). Thus, blood pressure can be measured frequently without placing stress on the subject's body as with ABPM.

### (Problem 1)

In order to accurately diagnose conditions caused by hypertension, home blood pressure measurement requires that the conditions under which blood pressure is measured daily be as constant as possible. Guidelines for the Management of Hypertension 2019 regarding the timing of measurements recommended by healthcare professionals for their patients include the following.

The recommended timing to take measurements in the morning is at a point in time within an hour after waking, after urinating, before eating, and before taking medication. The recommended timing to take measurements in the evening is before going to bed.

When the user who is to measure their blood pressure does so at a recommended timing, more accurate results can be obtained. However, with conventional systems, it can be difficult to take blood pressure measurements at a recommended timing due to reasons such as the user forgetting to measure their blood pressure in the morning, or remembering after breakfast that they were supposed to measure their blood pressure and then consequently performing the measurement after breakfast.

### (Problem 2)

Although the cuffless blood pressure monitor described above can prevent the user from forgetting to measure their blood pressure, in day to day life, the times of day that correspond to the recommended timings may vary. Stated differently, the time of day at which the individual wakes up, urinates, eats breakfast, takes medications, and goes to bed may not necessarily be at the same time each day, but may vary depending on the time of day the user is active.

FIG. 7 illustrates an example of a case in which a plurality of blood pressure measurements were taken at predetermined time intervals during a day.

As illustrated in FIG. 7, for example, even if blood pressure is measured using a cuffless blood pressure monitor every 30 minutes every day, it is difficult to identify measurements taken at the recommended timing among the numerous measurements. Stated differently, it is difficult to identify measurements taken at time t1, which is a point in time after waking up and urinating and before eating breakfast.

### (Problem 3)

In addition to regularly measuring blood pressure, it is also useful to record and manage a log of what the user has consumed in order to improve lifestyle-related diseases. For example, it is considered important to reduce the amount of salt in consumed foods in order to reduce symptoms in hypertensive patients. For example, the Guidelines for the Management of Hypertension 2019 states a salt intake target of less than 6 grams per day. To reduce daily salt intake, it is useful to keep track of salt intake by managing daily food consumption.

For example, the input of food consumption details at home is performed by AI-based image recognition on an image of a meal taken using the app mentioned above to estimate the dishes of a meal. This reduces the hassle of inputting information, but the vast variety of meals means that dish estimation accuracy is still low. Therefore, when the user is asked to select a dish from among the candidates listed from the dish estimation results, there are often no candidates for the user to select, and the user may need to make adjustments for accurate input. Accordingly, the input of food consumption details is still a cumbersome process.

Another challenge is that salt content, an important indicator in hypertension, cannot be determined from images alone. In typical salt content estimation methods, the amount of salt in a dish estimated from an image or selected by a user is assumed based on the typical seasoning of that dish. However, the actual salt content can vary greatly depending on the seasoning and amount, leading to cases where the estimated salt content may deviate from the actual salt content.

On the other hand, if salt intake is to be determined accurately, it is necessary to collect all the urine excreted by a person and determine the amount and composition of urine, which places a heavy burden on the patient and others.

Furthermore, there is a general tendency to neglect entering food consumption information for snacks between meals such as, such as snacks eaten outside of between breakfast, lunch, and dinner. As a result, there may be instances where the food consumption information for snacks is omitted.

Thus, there is a need for a way to easily input what was consumed.

### (Problem 4)

Meal content is important in improving symptoms in hypertensive patients, but it is known that individual patients have different meal content requirements that affect their blood pressure. Therefore, it is very useful to make patients aware of which meal contents contributed to their elevated blood pressure, in order to encourage them to change their own dietary behavior.

Conventional management apps that manage the blood pressure measurement results and food consumption logs have functions to manage and visualize the blood pressure measurement results and the food consumption logs. However, since the relationship between the blood pressure measurement results and the consumed food is unknown, users have difficulty understanding what food contributed to the increase in blood pressure.

In order to overcome the above Problems 1 and 2, the inventors have discovered a lifestyle improvement system, etc., that can support taking blood pressure measurements at appropriate timings.

A lifestyle improvement system according to a first aspect of the present disclosure includes: an obtainer that obtains wake-up information about an act of waking up by a user and breakfast preparation information about an act of preparing breakfast by the user; a determiner that determines an optimal timing for blood pressure measurement of the user based on the wake-up information and the breakfast preparation information; and a notifier that notifies timing information regarding the optimal timing determined by the determiner.

With this, the lifestyle improvement system can support the user to take blood pressure measurement at appropriate timings since it notifies the user with timing information regarding the optimal timing for the user to measure their blood pressure, which is determined based on the wake-up information and breakfast preparation information.

A lifestyle improvement system according to a second aspect of the present disclosure is the lifestyle improvement system according to the first aspect, wherein the optimal timing is included in a period from after the user wakes up to when the user starts the act of preparing breakfast.

This can support blood pressure measurement during a period of low blood pressure variability.

A lifestyle improvement system according to a third aspect of the present disclosure is the lifestyle improvement system according to the first aspect, wherein the obtainer further obtains urination information about urination by the user, and the determiner further determines the optimal timing based on the urination information.

With this, the lifestyle improvement system can support the user to take blood pressure measurement at more appropriate timings since it notifies the user with timing information regarding the optimal timing for the user to measure their blood pressure, which is determined based on the wake-up information, urination information, and breakfast preparation information.

A lifestyle improvement system according to a fourth aspect of the present disclosure is the lifestyle improvement system according to the third aspect, wherein the optimal timing is included in a period from after the user urinates to when the user starts the act of preparing breakfast.

This can support blood pressure measurement during a period of even lower blood pressure variability.

A lifestyle improvement system according to a fifth aspect of the present disclosure is the lifestyle improvement system according to any one of the first through fourth aspects, further including: an informer that prompts the user to measure blood pressure, based on the timing information.

With this, the lifestyle improvement system can support the user to take blood pressure measurement at appropriate timings since it can prompt the user to take a blood pressure measurement at the optimal timing for the user to measure their blood pressure. A lifestyle improvement system according to a sixth aspect of the present disclosure is the lifestyle improvement system according to any one of the first through fifth aspects, further including: a measurer that measures a blood pressure of the user, based on the timing information.

This allows the user to take a blood pressure measurement at the optimal timing for the user to measure their blood pressure.

A lifestyle improvement system according to a seventh aspect of the present disclosure is the lifestyle improvement system according to any one of the first through sixth aspects, further including: storage that stores each of one or more results of the blood pressure measurement of the user in association with a time of obtainment of the result; and an extractor that extracts, from the one or more results, a result of a blood pressure measurement taken at the optimal timing for blood pressure measurement of the user, based on the timing information.

With this, the lifestyle improvement system can identify the result of a blood pressure measurement taken at the appropriate timing since it can identify the optimal timing for the user to measure their blood pressure.

A lifestyle improvement system according to an eighth aspect of the present disclosure is the lifestyle improvement system according to any one of the first through seventh aspects, wherein the obtainer further obtains bed-time information about an act of going to bed by the user and history information indicating a history of blood pressure measurement by the user, the determiner further determines whether the user has taken a blood pressure measurement in a period of a predetermined amount of time ending when the user goes to bed, based on the bed-time information and the history information, and the notifier notifies the timing information when the determiner determines that the user has not taken the blood pressure measurement in the period.

With this, the lifestyle improvement system can support the user to take blood pressure measurement at an appropriate timing before bed since it notifies the user with timing information when the user has not taken their blood pressure measurement at the optimal timing before going to bed for the user to measure their blood pressure, based on the bed-time information and the history information.

A lifestyle improvement system according to a ninth aspect of the present disclosure includes: an obtainer that obtains bed-time information about an act of going to bed by a user and history information indicating a history of blood pressure measurement by the user; a determiner that determines whether the user has taken a blood pressure measurement in a period of a predetermined amount of time ending when the user goes to bed, based on the bed-time information and the history information; and a notifier that notifies the user with timing information regarding an optimal timing for blood pressure measurement when the determiner determines that the user has not taken the blood pressure measurement in the period.

With this, the lifestyle improvement system can support the user to take blood pressure measurement at an appropriate timing before bed since it notifies the user with timing information when the user has not taken their blood pressure measurement at the optimal timing before going to bed, based on the bed-time information and the history information.

A lifestyle improvement system according to a tenth aspect of the present disclosure is the lifestyle improvement system according to the eighth or ninth aspect, further including: an informer that prompts the user to measure blood pressure, based on the timing information.

With this, the lifestyle improvement system can support the user to take a blood pressure measurement at am appropriate timing before going to bed since it can prompt the user to take a blood pressure measurement at the optimal timing before going to bed.

A lifestyle improvement system according to an eleventh aspect of the present disclosure is the lifestyle improvement system according to any one of the eighth through tenth aspects, further including: a measurer that measures a blood pressure of the user, based on the timing information.

This allows the user to take a blood pressure measurement at the optimal timing at bedtime.

A lifestyle improvement system according to a twelfth aspect of the present disclosure is the lifestyle improvement system according to any one of the eighth through eleventh aspects, further including: storage that, when the determiner determines that the user has taken the blood pressure measurement in the period, stores a result of the blood pressure measurement taken in the period as a result obtained at the optimal timing for blood pressure measurement.

With this, the lifestyle improvement system can identify the result of a blood pressure measurement taken at an appropriate timing before going to bed since it can identify the optimal timing for the user to measure their blood pressure before going to bed.

A portable terminal according to a thirteenth aspect of the present disclosure is connected to a server over a network. The server includes: an obtainer that obtains wake-up information about an act of waking up by a user and breakfast preparation information about an act of preparing breakfast by the user; a determiner that determines an optimal timing for blood pressure measurement of the user based on the wake-up information and the breakfast preparation information; and a notifier that notifies the portable terminal with timing information regarding the optimal timing determined by the determiner. The portable terminal includes: a receiver that receives the timing information; and an informer that informs the user of an optimal timing to measure blood pressure, based on the timing information.

With this, the portable terminal can support the user to take blood pressure measurement at appropriate timings since it presents the user with the optimal timing for the user to measure their blood pressure, which is determined based on the wake-up information and breakfast preparation information.

A portable terminal according to a fourteenth aspect of the present disclosure is connected to a server over a network. The server includes: an obtainer that obtains bed-time information about an act of going to bed by a user and history information indicating a history of blood pressure measurement by the user; a determiner that determines whether the user has taken a blood pressure measurement in a period of a predetermined amount of time ending when the user goes to bed, based on the bed-time information and the history information; and a notifier that notifies the user with timing information regarding an optimal timing for blood pressure measurement when the determiner determines that the user has not taken the blood pressure measurement in the period. The portable terminal includes: a receiver that receives the timing information; and an informer that informs the user of an optimal timing to measure blood pressure, based on the timing information.

With this, the portable terminal can support the user to take blood pressure measurement at an appropriate timing before bed since it presents the user with a timing when the user has not taken their blood pressure measurement at the optimal timing before going to bed, based on the bed-time information and the history information.

A control method according to a fifteenth aspect of the present disclosure is a control method of a lifestyle improvement system including one or more processors and an informer, and includes: obtaining, by the one or more processors, wake-up information about an act of waking up by a user and breakfast preparation information about an act of preparing breakfast by the user; and informing, by the informer, the user of timing information regarding an optimal timing for blood pressure measurement of the user, determined based on the wake-up information and the breakfast preparation information.

With this, the lifestyle improvement system can support the user to take blood pressure measurement at appropriate timings since it notifies the user with timing information regarding the optimal timing for the user to measure their blood pressure, which is determined based on the wake-up information and breakfast preparation information.

A control method according to a seventeenth aspect of the present disclosure is a control method of a lifestyle improvement system including one or more processors and an informer, and includes: obtaining, by the one or more processors, bed-time information about an act of going to bed by a user and history information indicating a history of blood pressure measurement by the user; and informing, by the informer, the user of timing information regarding an optimal timing for blood pressure measurement when it is determined, based on the bed-time information and the history information, that the user has not taken a blood pressure measurement in a period of a predetermined amount of time ending when the user goes to bed.

With this, the lifestyle improvement system can support the user to take blood pressure measurement at an appropriate timing before bed since it notifies the user with timing information when the user has not taken their blood pressure measurement at the optimal timing before going to bed, based on the bed-time information and the history information.

These general or specific aspects may be implemented as a device/apparatus, an integrated circuit, a computer program, or a computer-readable recording medium such as a CD-ROM, or any combination thereof.

Hereinafter, embodiments will be described in detail with reference to the drawings, but unnecessarily detailed descriptions may be omitted. For example, detailed descriptions of well-known matters or descriptions of elements that are substantially the same as previously described elements may be omitted. This is to avoid redundancy and provide easily read descriptions for those skilled in the art.

Note that the inventors have provided the appended drawings and following description for the purpose of facilitating sufficient understanding of the present disclosure by those skilled in the art. Accordingly, the appended drawings and following description are not intended to limit the scope of the claims.

### Embodiment

### Configuration

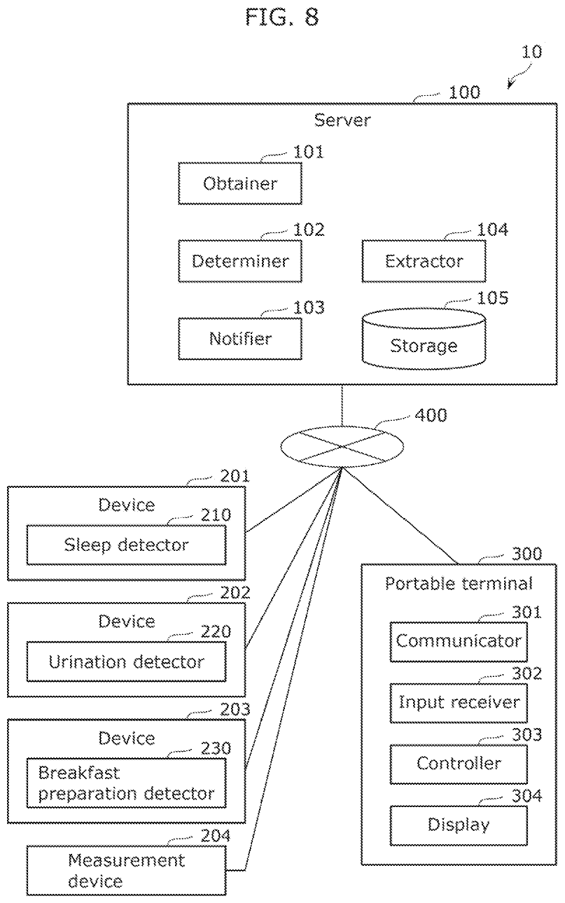

FIG. 8 illustrates one example of a functional block diagram of a lifestyle improvement system according to an embodiment.

As illustrated in FIG. 8, lifestyle improvement system 10 includes server 100 devices 201 through 203, measurement device 204, and portable terminal 300. Server 100, devices 201 through 203, measurement device 204, and portable terminal 300 are communicably connected via network 400. Network 400 may be a general-purpose line, such as the Internet, or a dedicated line. Note that devices 201 through 203 and measurement device 204 may be provided in a residence of the user, for example. Portable terminal 300 is a terminal carried by the user.

### (Devices)

Each of devices 201 through 203 is a home appliance or sensor device connected to network 400.

Device 201 includes sleep detector 210 that detects the user's sleep state. Device 201 is an Internet of Things (IoT) device with the ability to communicate with network 400. Device 201 may be, for example, a wearable terminal worn by the user, a sensor device or home appliance including a radio wave sensor that senses the user's movement in the space above the user's bed (sleeping area), or a sensor device that detects the user's pulse wave in the space above the bed. When device 201 is a wearable terminal, sleep detector 210 may detect changes in the user's body movements measured by the six-axis sensors included in the wearable terminal (the triaxial acceleration sensor and the triaxial angular rate sensor) as the sleep state, and, alternatively may detect changes in the user's heartbeat or pulse wave measured by a rate sensor included in the wearable terminal as the sleep state. When device 201 is a sensor device or home appliance including a radio wave sensor provided bedside, sleep detector 210 may detect changes in the user's body movements measured by the radio wave sensor as the sleep state. Sleep detector 210 detects the user's sleep state and transmits sleep information indicating the detected sleep state to server 100 via network 400. Since it can be estimated from sleep information that the user has woken up and that the user has gone to bed, sleep information can include wake-up information about an act of waking up by the user and bed-time information about an act of going to bed by the user.

Device 202 includes urination detector 220 that detects user urination. Device 202 is an IoT device with the ability to communicate with network 400. Device 202 may be, for example, a sensor device including a motion sensor provided in a restroom (i.e., a space where a toilet bowl is located), and, alternatively, may be a toilet bowl including a motion sensor for opening and closing the lid of the toilet bowl. In cases in which a plurality of people live in a residence with a restroom, identification of who has entered the restroom may be carried out by communicating with a portable terminal or wearable terminal of the user. The identification may be done by facial recognition or by using physical characteristics such as weight, height, etc. Urination detector 220 detects a person in the restroom, and transmits the detection result of the person in the restroom to server 100 via network 400 as urination information. In cases in which identification of who has entered the restroom is carried out, urination detector 220 may transmit information in which the identification result is associated with the detection result to server 100 via network 400 as the urination information. Urination information is information about the user's urination, i.e., is information for determining whether the user has urinated or not.

Device 203 includes breakfast preparation detector 230 that detects an act of preparing breakfast by the user. Device 203 is an IoT device with the ability to communicate with network 400. Device 203 is a home appliance in the kitchen, such as a refrigerator, microwave oven, toaster, electric pot, electric kettle, gas stove, or induction cooktop. For example, if device 203 is a refrigerator, breakfast preparation detector 230 detects the opening and closing of the refrigerator door as the act of preparing breakfast. For example, if device 203 is a microwave oven, toaster, electric pot, electric kettle, gas stove, induction cooktop, etc., breakfast preparation detector 230 detects the turning on of power and the starting of operation as the act of preparing breakfast. Breakfast preparation detector 230 transmits the detection result to server 100 via the network as breakfast preparation information.

Thus, if devices 201 through 203 are IoT-enabled, it is possible to grasp the usage status (operating status) of each home appliance or sensor device, and the usage status can be transmitted to and stored in server 100 that manages the home appliances or portable terminal 300 such as a smartphone that is connected to a home appliance via Bluetooth or the like. Even if neither the home appliance nor the sensor device is IoT-enabled, the usage status of the home appliance may be transmitted to server 100 or portable terminal 300 by using a sensor device that includes a communication function and is inserted between the home appliance and the wall outlet to measure the power consumption of the home appliance.

Using the sleep information, the urination information, and the breakfast preparation information obtained by a plurality of devices 201 through 203, such as device 201 which detects the sleep state, device 202 which detects the usage status of a restroom, and device 203 which detects meal preparation, it is possible to estimate a period suitable for morning blood pressure measurement (i.e., within one hour after waking; after urination; before eating or taking medication). If it is found that blood pressure has not been measured during the period suitable for morning blood pressure measurement, for example, at a time when a kitchen appliance is detected to be in use (i.e., when it is presumed that breakfast has not yet been eaten), it is possible to prompt those who need to take a blood pressure measurement to do so by a notification from portable terminal 300, by an LED on the blood pressure monitor, by a notification sound output from the blood pressure monitor, by a notification prompting blood pressure measurement displayed on the display of a home appliance, or by a notification via voice or a beep from home appliance.

### (Measurement Device)

Measurement device 204 is a device that has the function of measuring the user's blood pressure. Measurement device 204 is an IoT device connected to network 400. Measurement device 204 transmits the result of the blood pressure measurement to server 100 via network 400. Measurement device 204 may, for example, transmit the result of the blood pressure measurement to server 100 each time a blood pressure measurement is taken, and, alternatively, may transmit the results of one or more unsent blood pressure measurements obtained at predetermined intervals to server 100.

### (Server)

Server 100 is a cloud server that provides information for supporting the improvement of a user's lifestyle. Server 100 includes obtainer 101, determiner 102, notifier 103, extractor 104, and storage 105. Server 100 includes a processor and memory. The functions of server 100 may be realized by a processor executing a program stored in memory.

Obtainer 101 is a communication interface (IF) that obtains various information from devices 201 through 203, measurement device 204, and portable terminal 300 by communicating with devices 201 through 203, measurement device 204, and portable terminal 300 via network 400. Obtainer 101 obtains the user's sleep information from device 201 via network 400. Stated differently, obtainer 101 obtains the user's wake-up information. Obtainer 101 also obtains the user's bed-time information. Obtainer 101 further obtains breakfast preparation information about an act of preparing breakfast by the user from device 203 via network 400. Obtainer 101 may obtain urination information about urination by the user from device 202 via network 400. Obtainer 101 may also obtain history information indicating a history of blood pressure measurement by the user from storage 105.

Determiner 102 determines an optimal timing for the user's blood pressure measurement based on the sleep information and the breakfast preparation information. More specifically, determiner 102 estimates the time at which the user wakes up based on the sleep information. Determiner 102 also estimates the start of the act of preparing the user's breakfast based on the breakfast preparation information. Determiner 102 thus determines that the period between the time the user wakes up and the time the user starts the act of preparing breakfast is the optimal timing for the user's blood pressure measurement.

Determiner 102 may determine the optimal timing for the user's blood pressure measurement based further on the urination information. More specifically, determiner 102 may determine that the period from the time the user urinates to the time the user starts the act of preparing breakfast is the optimal timing for the user's blood pressure measurement.

The wake-up information included in the sleep information may be information indicating the wake-up alarm setting or notification status on a smartphone, smart speaker, etc. In such cases, determiner 102 may estimate the time that the user wakes up based on the wake-up alarm setting or notification status.

Notifier 103 notifies timing information regarding the optimal timing for the user's blood pressure measurement as determined by determiner 102. For example, notifier 103 notifies portable terminal 300 or measurement device 204 with the timing information to notify the user that the time of the notification of the timing information is an appropriate timing for blood pressure measurement. For example, notifier 103 notifies the timing information to extractor 104 to notify the user that the time indicated in the timing information is an appropriate timing for blood pressure measurement.

Determiner 102 may determine whether the user has measured their blood pressure in a period of a predetermined amount of time ending when the user goes to bed, based on the bed-time information and the history information. The history information is information indicating a history of blood pressure measurement by the user. The history information associates one or more results of user blood pressure measurements with one or more measurement times each corresponding to one or more results. Each of the one or more measurement times is the time at which the blood pressure measurement was taken, and corresponds to one or more results. The history information is stored in storage 105. As used herein, “going to bed” and functionally equivalent phrases mean that the user lies down on a bed (bedding) to sleep, and includes cases in which the user is not necessarily asleep even after getting in bed.

In such cases, notifier 103 may notify the timing information if determiner 102 determines that blood pressure has not been measured in the period of a predetermined amount of time ending when the user goes to bed. In such cases as well, for example, notifier 103 notifies portable terminal 300 or measurement device 204 with the timing information to notify the user that the time of the notification of the timing information is an appropriate timing for blood pressure measurement. For example, notifier 103 notifies the timing information to extractor 104 to notify the user that the time indicated in the timing information is an appropriate timing for blood pressure measurement.

If determiner 102 determines that blood pressure has been measured in the period of a predetermined amount of time ending when the user goes to bed, a result of a blood pressure measurement taken in that period may be stored in storage 105 as a result obtained at the optimal timing for blood pressure measurement.

Based on the timing information obtained from notifier 103, extractor 104 may extract the results of one or more blood pressure measurements that were taken at an appropriate timing for the blood pressure measurement indicated in the timing information, from among one or more results of blood pressure measurements included in the history information stored in storage 105. For example extractor 104 may extract, from among one or more results, a result obtained by taking a blood pressure measurement in the period from the time the user wakes up (or urinates) to the time at which the user starts the act of preparing breakfast. For example, extractor 104 may extract, from among one or more results, a result obtained by taking a blood pressure measurement in the period of a predetermined amount of time ending when the user goes to bed.

Storage 105 may store sleep information, urination information, and breakfast preparation information. Storage 105 may store the history information. Storage 105 may store, as food consumption log information, each of one or more first food consumption logs in association with the timing at which the user consumed the food and/or drink indicated in the first food consumption log. Each of the one or more first food consumption logs indicates the food and/or beverage consumed by the user. Storage 105 may store sleep information, urination information, breakfast preparation information, history information, and food consumption log information associated with user ID that identifies the user. This information stored in storage 105 is managed by periodically updating them with the latest per-user information.

The sleep information, urination information, breakfast preparation information, history information, and food consumption log information may be assigned a device ID for the device that generated the respective information. Associations between device IDs and user IDs may be managed in storage 105. User IDs corresponding to each of the sleep information, urination information, breakfast preparation information, history information, and food consumption log information may be identified from the device ID assigned to each item of information, based on the associations between device IDs and user IDs. The sleep information, urination information, breakfast preparation information, history information, and food consumption log information may be assigned a user ID obtained by inputting the user ID into the device that generated the respective information.

### (Portable Terminal)

Portable terminal 300 is a terminal such as a smartphone or tablet possessed by a user.

Portable terminal 300 includes communicator 301, input receiver 302, controller 303, and display 304. Portable terminal 300 includes a processor and memory. The functions of portable terminal 300 may be realized by the processor executing a program stored in the memory.

Communicator 301 exchanges information with server 100 via network 400. Communicator 301 may also exchange information with devices 201 through 203 and measurement device 204 via network 400. The various information obtained at devices 201 through 203 and measurement device 204 may be transmitted to server 100 via communicator 301 of portable terminal 300. In such cases, communicator 301 may communicate directly with devices 201 through 203 and measurement device 204 via wireless communication such as via Bluetooth or Wi-Fi (registered trademark) directly, without going through network 400. Communicator 301 receives timing information from server 100. Communicator 301 may also receive history information from server 100. Communicator 301 may transmit food consumption information indicating the user's consumed food entered by the user to server 100. Communicator 301 may transmit a request for one or more first food consumption logs and receive food consumption log information indicating the one or more first food consumption logs. The one or more first food consumption logs are stored in storage 105 of server 100. The one or more first food consumption logs contain one or more instances of food consumption information regularly transmitted from portable terminal 300. The one or more first food consumption logs are associated with user IDs and managed on a per user basis.

Input receiver 302 is an input interface (IF) that accepts inputs from the user. Input receiver 302 may accept a selection of one result among one or more blood pressure measurement results. For example, the selection may be made based on user input, made via the touch panel included in portable terminal 300, of a result among one or more of results included in the user interface (UI) displayed on display 304. Input receiver 302 may accept the selection of a dish for a meal that has been consumed (or is to be consumed) from a plurality of dish candidates. For example, the selection may be made based on user input, made via the touch panel included in portable terminal 300, of a dish among one or more of dishes candidates included in the user interface (UI) displayed on display 304.

When communicator 301 receives the timing information, controller 303 informs the user so as to prompt the user to measure their blood pressure based on the timing information. Stated differently, controller 303 is one example of the informer.

Controller 303 identifies, among the one or more first food consumption logs, one or more second food consumption logs that are associated with a second timing included in period of a predetermined amount of time ending at a first timing at which one result was obtained, and displays the identified one or more second food consumption logs on display 304. For example, controller 303 displays a UI on display 304 that includes the one or more second food consumption logs.

The process of identifying one or more second food consumption logs from the one or more first food consumption logs by controller 303 may be performed by server 100. In such cases, information indicating one result selected via input receiver 302 is transmitted to server 100, and server 100 may, based on this information, identify one or more second food consumption logs from the one or more first food consumption logs and transmit information indicating the one or more second food consumption logs to portable terminal 300. Portable terminal 300 may then display the information indicating the received one or more second food consumption logs on display 304.

Display 304 is a display that displays images. Display 304 displays images and UI generated by controller 303.

### (Specific Example of Device)

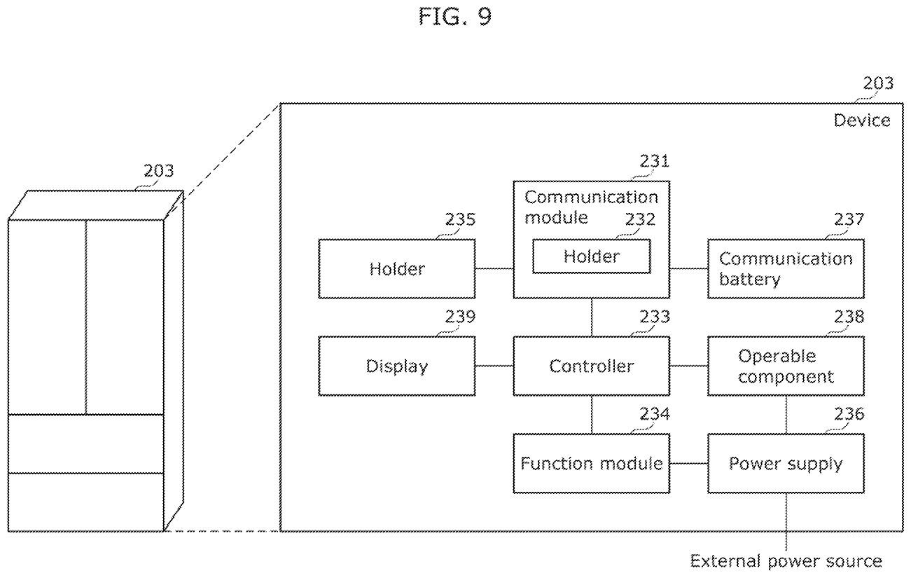

FIG. 9 illustrates the configuration of a device when the device is a refrigerator capable of communication.

As illustrated in FIG. 9, device 203 includes communication module 231, controller 233, function module 234, holder 235, power supply 236, communication battery 237, operable component 238, and display 239.

Communication module 231 connects to server 100 that manages device 203 via a specific network. For example, communication module 231 is a communication module for wireless communication such as wireless LAN (for example, Wi-Fi) or long-distance wireless communication such as LPWA. Communication module 231 includes holder 232 that holds the module ID of the communication module. In cases in which communication module 231 includes a plurality of communication modules that employ different communication methods, holder 232 holds the module ID of each of the communication modules.

Controller 233 controls the operation of device 203 when device 203 is powered on. More specifically, controller 233 controls the operation of device 203 by controlling function module 234. Controller 233 may also generate state information for device 203 and transmit the generated state information to server 100 using communication module 231. Specifically, controller 233 may obtain the power state of power supply 236 of device 203, which indicates whether the power is on or off, and generate state information including the power state, and may generate state information including functional information indicating functions carried out by function module 234. Controller 233 may obtain the state of the refrigerator door, i.e., whether the door is open or closed, and transmit state information including the state of the refrigerator door to server 100 using communication module 231. The state information generated by controller 233 may include device ID, transmission date and time, etc. Controller 233 may also display an image on display 239 based on information received from server 100 via communication module 231.

Function module 234 is a module that carries out functions of device 203.

Holder 235 is a storage device that holds a unique ID for each device 203. Hereinafter, the transmission of state information to server 100 may be referred to as the “notification” of state information.

Power supply 236 receives power from an external power source and supplies power to the elements included in device 203.

Communication battery 237 is a battery that provides power to drive communication module 231 and the like. Communication battery 237 may be a primary or secondary battery. This allows communication module 231 to transmit state information to server 100 via a base station even when device 203 is turned off. Stated differently, since communication module 231 can communicate with a base station regardless of whether device 203 is powered on or off, it can transmit state information to server 100 at any time.

Operable component 238 is an input device that accepts operations made by the user with respect to device 203. If device 203 includes one or more doors, etc., that open and close, such as a refrigerator, microwave oven, rice cooker, etc., operable component 238 may be these one or more doors.

Display 239 is a display device that displays various information as images.

Next, the configuration of device 203 will be described in detail using a refrigerator as an example.

Even though it is connected to the Internet as an IoT device, device 203, a refrigerator, is used as a home appliance and includes various modules to realize its original function as a home appliance. Assuming device 203 is a refrigerator, such modules include a compressor to cool the interior, a lighting system to illuminate the interior when the door is opened, and a sensor to measure the temperature or humidity inside the refrigerator. Such modules correspond to function modules 234. Large home appliances such as refrigerators or air conditioners are generally configured to be connected to an external power source via power supply 236.

In addition, it is common in recent years for home appliances to include a microcomputer or processor-based controller 233 to control various functions of convenience. For example, in a refrigerator with an ice-making function, a sensor installed in a special dish where ice is stored determines whether ice is made or not, and operates to make new ice. To perform these particular operations, control is carried out via the microcomputer or processor and software executed thereby.

Device 203 further includes display 239 for presenting various information to the user or operable component 238 for the user to perform complex operations.

A conventional display in a device is limited to the minimum necessary indication, such as indicating an abnormal state or whether the device is powered on or off, by using lamps or a display including few digits of numbers. Additionally, operation of conventional displays has been simple, with only a few buttons for quick freezing instructions or resetting in the event of an abnormality.

In contrast, device 203 includes a small touch panel display as operable component 238 and display 239, allowing for the display of more complex states and allowing for various settings to be configured.

In contrast to device 203, what characterizes IoT home appliances is communication module 231. Communication module 231 enables connection to the Internet via one or more of various communication methods, such as Wi-Fi or LTE. When a plurality of communication modules are installed, each communication module is assigned with an individual communication module ID, which, depending on the communication method, serves as a communication identifier, like a phone number in LTE, for example. By connecting to the Internet, various information collected by controller 233 can be transmitted to server 100, and, conversely, information needed to control device 203 can be obtained from server 100.

### (Specific Example of Portable Terminal)

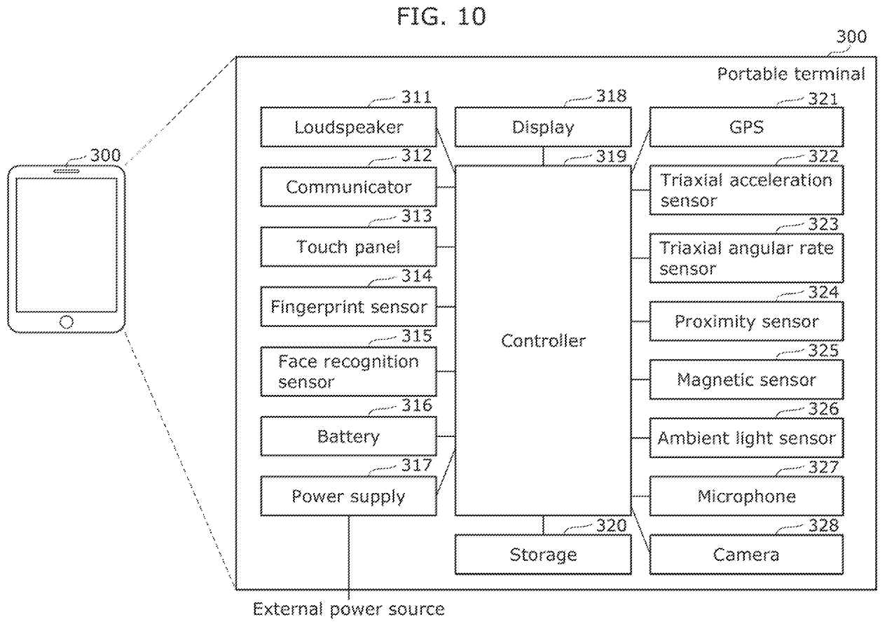

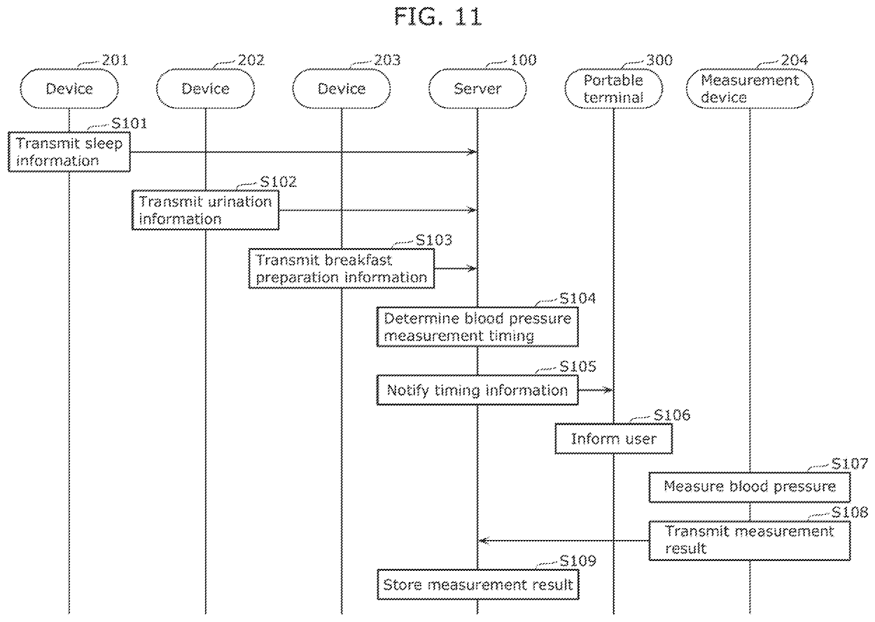

FIG. 10 illustrates the configuration of the portable terminal in detail. FIG. 10 schematically illustrates the block configuration of a smartphone as one example of the portable terminal.

As illustrated in FIG. 10, portable terminal 300 includes loudspeaker 311, communicator 312, touch panel 313, fingerprint sensor 314, face recognition sensor 315, battery 316, power supply 317, display 318, controller 319, storage 320, GPS 321, triaxial acceleration sensor 322, triaxial angular rate sensor 323, proximity sensor 324, magnetic sensor 325, ambient light sensor 326, microphone 327, and camera 328.

Controller 319 is capable of integrally controlling portable terminal 300 and includes a central processing unit (CPU) and a memory element (such as SRAM) (not illustrated). Display 318 is a display that displays text and graphics based on information received from controller 319. Storage 320 stores the operating system (OS) to be read and executed by controller 319, various application programs (generally referred to as smartphone apps), and various data used by various programs. Storage 320 includes a nonvolatile storage area.

Communicator 312 implements wireless communication functions such as long term evolution (LTE) and 5G, and is wirelessly connected to a base station of a cellular phone communication network (not illustrated) and connected to the internet via the base station. Communicator 312 may implement wireless communication functions via Bluetooth or wireless LAN (for example, Wi-Fi). Communicator 312 may be connected to the internet via Bluetooth or wireless LAN (for example, Wi-Fi) through other communication devices.

Touch panel 313 accepts input from the user to the UI displayed on display 318 and transmits signals based on the input (for example, which part of the screen is being touched and with what degree of pressure) to controller 319. GPS 321 measures the geographical position of portable terminal 300. Triaxial acceleration sensor 322 sets the X-, Y-, and Z-axes for portable terminal 300, and measures acceleration in each axis. Triaxial angular rate sensor 323 measures angular velocity in the direction of rotation for each axis. Proximity sensor 324 detects objects in close proximity (for example, a face approaching the smartphone). Magnetic sensor 325 detects the geomagnetic field and indicates the direction. Ambient light sensor 326 detects the ambient brightness of portable terminal 300. Microphone 327 collects ambient sounds and speech. Camera 328 captures images from the front of portable terminal 300, where display 318 is located, or from the rear on the opposite side. Loudspeaker 311 emits sound based on audio signals generated by controller 319. Fingerprint sensor 314 is used for user authentication, etc., and detects a person's fingerprint. Face recognition sensor 315 authenticates a person's face. Face recognition sensor 315 may be realized by combining an infrared camera, a projection illuminator, a dot projector, etc., with a group of sensors (a proximity sensor, a camera, and an ambient light sensor).

Power supply 317 receives a supply of power from an external power source. Battery 316 stores the external power received by power supply 317 or supplies the stored power to portable terminal 300 for operating.

### Operations Related to Blood Pressure Measurement

### First Example

Next, a first example of operations related to blood pressure measurement performed by lifestyle improvement system 10 will be described.

FIG. 11 is a sequence diagram illustrating a first example of operations related to blood pressure measurement performed by a lifestyle improvement system.

Device 201 transmits sleep information generated by detecting the user's sleep state to server 100 (S101).

Device 202 transmits urination information generated by detecting user urination to server 100 (S102).

Device 203 transmits the preparation information generated by detecting an act of preparing breakfast by the user to server 100 (S103).

Next, upon receiving the sleep information, the urination information, and the preparation information, server 100 determines the optimal timing for the user's blood pressure measurement based on the sleep information, the urination information, and the preparation information (S104).

Server 100 notifies portable terminal 300 with the timing information regarding the determined timing (S105). The timing information is used to notify that the timing of notification of the timing information is the appropriate timing for blood pressure measurement.

Based on the timing information, portable terminal 300 informs the user so as to prompt the user to measure their blood pressure (S106). Upon receiving the timing information, portable terminal 300 informs the user as described above without waiting for a predetermined period of time.

The user uses measurement device 204 to measure their blood pressure (S107).

After the blood pressure measurement is taken, measurement device 204 transmits the measurement result to server 100 (S108).

When server 100 receives the measurement result from measurement device 204, it stores the received measurement result in storage 105 (S109).

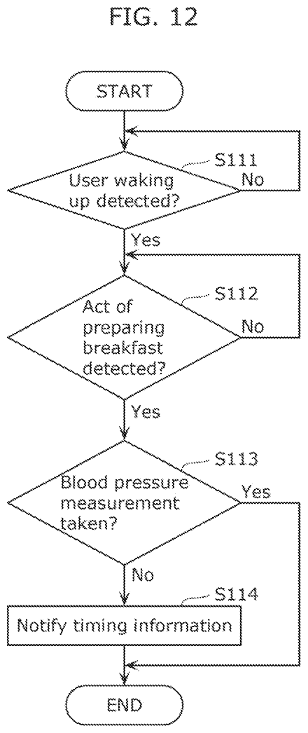

FIG. 12 is a flowchart illustrating the processes of determining the optimal timing for blood pressure measurement and notifying the timing information that are performed in the first example. Stated differently, FIG. 12 illustrates a specific example of steps S104 and S105. These processes are initiated when server 100 receives the sleep information and preparation information.

Server 100 detects whether or not the user has woken up at the current time based on the sleep information (S111).

If server 100 detects that the user has woken up at the current time (Yes in S111), it detects whether the user has started the act of preparing breakfast at the current time based on the preparation information (S112). If server 100 detects that the user has not woken up at the current time (No in S111), it returns to step S111. Stated differently, server 100 repeats step S111 until it detects that the user has woken up.

If server 100 detects that the act of preparing breakfast has started (Yes in S112), it determines whether a result of a blood pressure measurement conducted after the starting of the act of preparing breakfast is stored in storage 105 (S113). If server 100 detects that the act of preparing breakfast has not started (No in S112), it returns to step S112. Stated differently, server 100 repeats step S112 until it detects that the act of preparing breakfast has started.

If server 100 determines that a result of a blood pressure measurement conducted after the starting of the act of preparing breakfast is not stored in storage 105 (No in S113), it determines that the current time is the optimal timing for blood pressure measurement and notifies portable terminal 300 with timing information (S114). Note that if server 100 determines that a result of a blood pressure measurement conducted after the starting of the act of preparing breakfast is stored in storage 105 (Yes in S113), the process is terminated.

This allows portable terminal 300 to inform the user in step S106 so as to prompt the user to measure their blood pressure based on the timing information, thereby prompting the user to measure their blood pressure at the optimal timing for the user to measure their blood pressure.

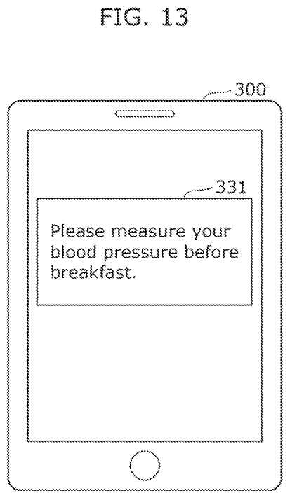

FIG. 13 is a diagram illustrating one example of informing the user so as to prompt the user to measure blood pressure.

Portable terminal 300 informs the user of the above by displaying message 331 prompting the user to measure his or her blood pressure, such as “please measure your blood pressure before breakfast”.

Note that if the presence or absence of a blood pressure measurement cannot be confirmed in step S113 (i.e., it cannot be determined whether or not a blood pressure measurement has been taken), portable terminal 300 may display the message “please measure your blood pressure before breakfast” to the user in step S106, regardless of whether or not a blood pressure measurement has been taken. Although such a notification is unnecessary if a blood pressure measurement has already been taken, it prevents the user from forgetting to measure their blood pressure.

Server 100 may also check the presence or absence of a blood pressure measurement in advance, hold a result of the check, and use the result to determine whether or not to prompt the user to take a blood pressure measurement when the start of breakfast preparation is detected. Stated differently, the order in which steps S112 and S113 are performed may be reversed, as long as the informing for prompting blood pressure measurement is performed when there is no blood pressure measurement and when the start of breakfast preparation is detected.

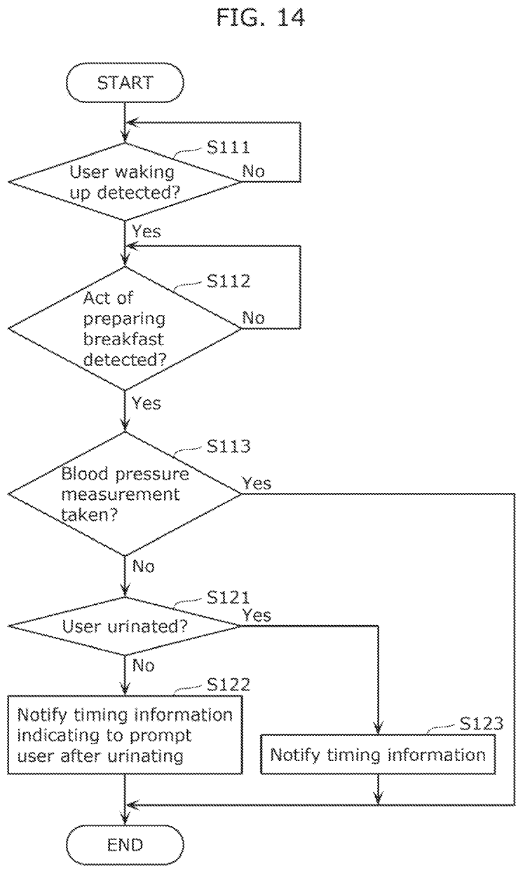

FIG. 14 is a flowchart illustrating another example of the processes of determining the optimal timing for blood pressure measurement and notifying the timing information. Stated differently, FIG. 12 illustrates a specific example of steps S104 and S105. Description of these processes will focus on the new steps S121 through S123 since steps S111 through S113 are the same as in FIG. 12. These processes are initiated when server 100 receives the sleep information, the preparation information, and the urination information.

If server 100 determines that a result of a blood pressure measurement conducted after the starting of the act of preparing breakfast is not stored in storage 105 (No in S113), it determines whether the user has urinated after waking up (S121). Note that if server 100 determines that a result of a blood pressure measurement conducted after the starting of the act of preparing breakfast is stored in storage 105 (Yes in S113), the process is terminated.

If server 100 determines that the user has not urinated after waking up (No in S121), it notifies portable terminal 300 with timing information indicating to prompt the user to take a blood pressure measurement after urinating (S122). This allows portable terminal 300 to inform the user in step S106 so as to prompt the user to measure their blood pressure after urinating based on the timing information, thereby prompting the user to measure their blood pressure at the optimal timing for the user to measure their blood pressure.

If server 100 determines that the user has urinated after waking up (Yes in S121), it notifies portable terminal 300 with timing information indicating to prompt the user to take a blood pressure measurement (S123). This allows portable terminal 300 to inform the user in step S106 so as to prompt the user to measure their blood pressure based on the timing information, thereby prompting the user to measure their blood pressure at the optimal timing for the user to measure their blood pressure.

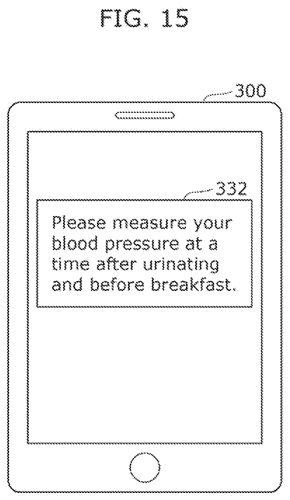

FIG. 15 is a diagram illustrating one example of informing the user so as to prompt the user to measure blood pressure after urinating.

Portable terminal 300 informs the user of the above by displaying message 332 prompting the user to measure his or her blood pressure after urinating, such as “please measure your blood pressure at a time after urinating and before breakfast”.

Note that if whether urination cannot be confirmed in step S121 (i.e., it cannot be determined whether or not the user has urinated), portable terminal 300 may display the message “please measure your blood pressure at a time after urinating and before breakfast” to the user in step S106, regardless of whether or not the user has urinated. This makes it possible to prompt the user to measure their blood pressure after urinating.

The availability of urination detection and the availability of blood pressure measurement detection may be managed by portable terminal 300 or server 100 based on the availability of an environment in which the detections can be carried out.

Server 100 may also check for urination in advance, hold a result of the check, and use the result to determine the content of the notification when the start of breakfast preparation is detected.

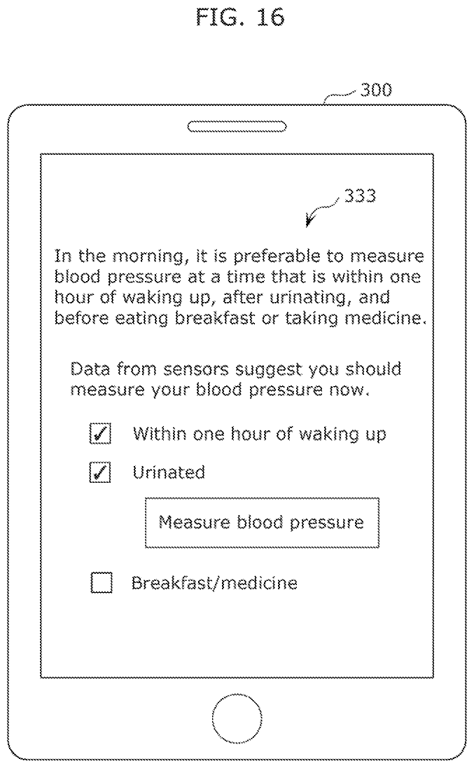

FIG. 16 illustrates one example of a UI that accepts input of a timing at which a blood pressure measurement was taken.

UI 333 may include a check list that accepts inputs indicating whether the user took their blood pressure measurement at a time that is within an hour after waking up, after urinating, and before eating breakfast and taking medication. With this, if a blood pressure measurement is taken after breakfast, for example, supplemental information indicating this timing is recorded. Accordingly, a physician can later reference this supplemental information when deciding whether to use a log of measurement results including such a result in their diagnosis process.

Each item in the check list may be automatically populated based on detection results by various sensors, and, alternatively may be populated with initial values in UI 333.

### Second Example

Next, a second example of operations related to blood pressure measurement performed by lifestyle improvement system 10 will be described.

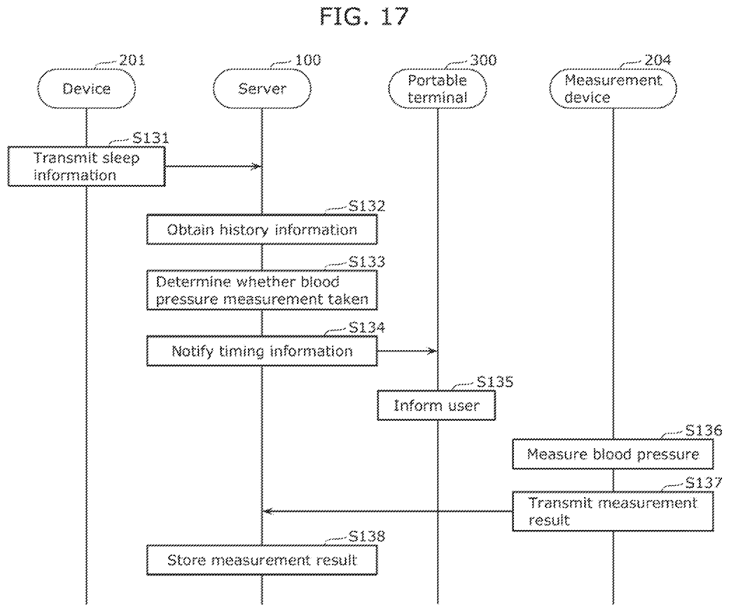

FIG. 17 is a sequence diagram illustrating a second example of operations related to blood pressure measurement performed by a lifestyle improvement system.

Device 201 transmits sleep information generated by detecting the user's sleep state to server 100 (S131).

Next, server 100 obtains the history information from storage 105 (S132). Stated differently, server 100 obtains the sleep information and the history information through the execution of steps S131 and S132.

Server 100 determines whether the user has measured their blood pressure in a period of a predetermined amount of time ending when the user goes to bed, based on the sleep information and the history information (S133).

Server 100 notifies portable terminal 300 with the timing information regarding the determined timing (S134).

Based on the timing information, portable terminal 300 informs the user so as to prompt the user to measure their blood pressure (S135).

The user uses measurement device 204 to measure their blood pressure (S136).

After the blood pressure measurement is taken, measurement device 204 transmits the measurement result to server 100 (S137).

When server 100 receives the measurement result from measurement device 204, it stores the received measurement result in storage 105 (S138).

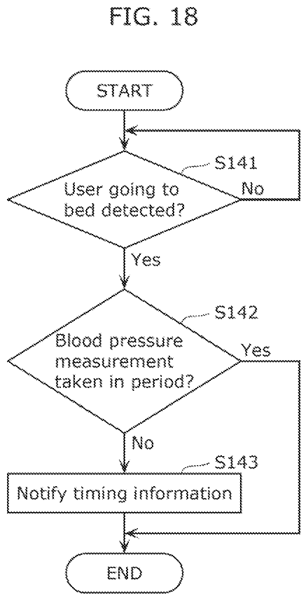

FIG. 18 is a flowchart illustrating the processes of determining the optimal timing for blood pressure measurement and notifying the timing information that are performed in the second example. Stated differently, FIG. 12 illustrates a specific example of steps S133 and S134. These processes are initiated when server 100 receives the sleep information.

Server 100 detects whether or not the user has gone to bed at the current time based on the sleep information (S141).

If server 100 detects that the user has gone to bed at the current time (Yes in S141), it determines whether a result of a blood pressure measurement taken by the user in a period of a predetermined amount of time ending when the user goes to bed is stored in storage 105 (S142). If server 100 detects that the user has not gone to bed at the current time (No in S141), it returns to step S141. Stated differently, server 100 repeats step S141 until it detects that the user has gone to bed.

If server 100 determines that a result of a blood pressure measurement taken by the user in a period of a predetermined amount of time ending when the user goes to bed is not stored in storage 105 (No in S142), it determines that the current time is the optimal timing for blood pressure measurement and notifies portable terminal 300 with timing information (S143). Note that if server 100 determines that a result of a blood pressure measurement taken by the user in a period of a predetermined amount of time ending when the user goes to bed is stored in storage 105 (Yes in S142), the process is terminated.

This allows portable terminal 300 to inform the user in step S135 so as to prompt the user to measure their blood pressure based on the timing information, thereby prompting the user to measure their blood pressure at the optimal timing for the user to measure their blood pressure.

### Third Example

Next, a third example of operations related to blood pressure measurement performed by lifestyle improvement system 10 will be described.

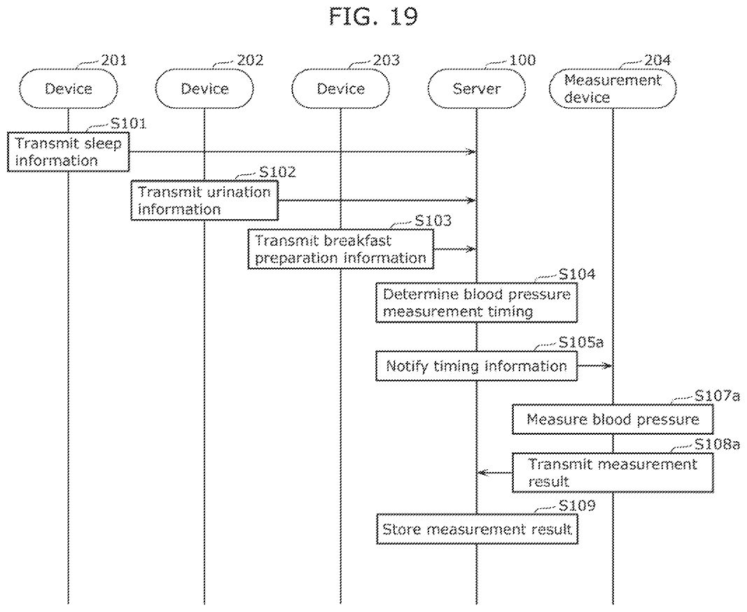

FIG. 19 is a sequence diagram illustrating a third example of operations related to blood pressure measurement performed by a lifestyle improvement system. Measurement device 204 in this case is a cuffless blood pressure measurement device, which is a wearable terminal worn by the user. Measurement device 204 is one example of the measurer.

Since steps S101 through S104 and S109 in the first example of the operation of the lifestyle improvement system are the same in the third example, description will focus on the different steps S105a, S107a, and S108a.

After step S104, server 100 notifies measurement device 204 with the timing information regarding the determined timing (S105a). The timing information is used to notify that the timing of notification of the timing information is the appropriate timing for blood pressure measurement.

Measurement device 204 measures the user's blood pressure based on the timing information (S107a). Upon receiving the timing information, measurement device 204 performs the above-described blood pressure measurement without waiting for a predetermined period of time.

After the blood pressure measurement is taken, measurement device 204 transmits the measurement result to server 100 (S108a).

When server 100 receives the measurement result from measurement device 204, it stores the received measurement result in storage 105 (S109).

### Fourth Example

Next, a fourth example of operations related to blood pressure measurement performed by lifestyle improvement system 10 will be described.

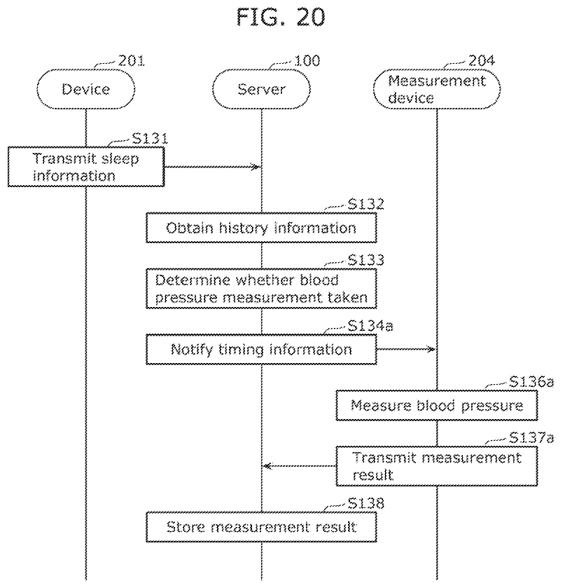

FIG. 20 is a sequence diagram illustrating a fourth example of operations related to blood pressure measurement performed by a lifestyle improvement system. Measurement device 204 in this case is a cuffless blood pressure measurement device, which is a wearable terminal worn by the user. Measurement device 204 is one example of the measurer.

Since steps S131 through S133 and S138 in the second example of the operation of the lifestyle improvement system are the same in the fourth example, description will focus on the different steps S134a, S136a, and S137a.

After step S133, server 100 notifies measurement device 204 with the timing information regarding the determined timing (S134a).

Measurement device 204 measures the user's blood pressure based on the timing information (S136a). Upon receiving the timing information, measurement device 204 performs the above-described blood pressure measurement without waiting for a predetermined period of time.

After the blood pressure measurement is taken, measurement device 204 transmits the measurement result to server 100 (S137a). When server 100 receives the measurement result from measurement device 204, it stores the received measurement result in storage 105 (S138).

### Operations Related to Input of Food Consumption Log Entries

Next, one example of operations related to input of food consumption log entries in lifestyle improvement system 10 will be described.

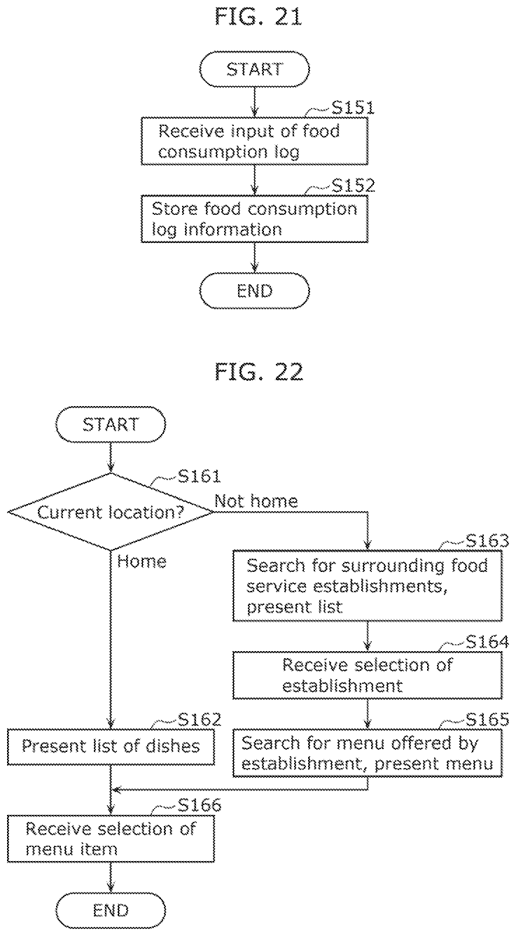

FIG. 21 is a flowchart illustrating one example of operations related to input of food consumption log entries in the lifestyle improvement system.

Portable terminal 300 receives an input of a food consumption log (S151). A specific example of step S151 will be given later.

Next, portable terminal 300 generates food consumption log information based on the received input and stores the generated food consumption log information (S152). Note that the food consumption log information may be transmitted to server 100 and stored in server 100.

### First Example

FIG. 22 is a flowchart illustrating a first example of a process of inputting a food consumption log.

When the user uses an app on portable terminal 300 to input a food consumption log, portable terminal 300 obtains the current location using GPS or other some means, compares the obtained current location with the location information of the user's home, and determines whether the current location is the user's home or not (S161).

If portable terminal 300 determines that the current location is the user's home (“home” in S161), it estimates that the meal being entered by the user was prepared in the home and presents the user with a list of dishes stored in the app or in server 100 connected via the internet (S162). In step S166 after this, a food consumption log is input by portable terminal 300 receiving the user's selection of an eaten (or to be eaten) dish. Other possible methods for inputting food consumption log entries include taking pictures or extraction from the browsing history of cooking recipes.

If portable terminal 300 determines that the current location is outside the home (“not home” in S161), it estimates that the meal being entered by the user is from a food service establishment, and searches for a restaurant or other food service establishment near the current location based on location information. Portable terminal 300 then presents the user with a list of potential food service establishments (S163). When presenting this list of food service establishment, portable terminal 300 may highlight establishments that the user has patronized to make the selection easier for the user.

Next, portable terminal 300 receives the user's selection of a food service establishment (S164).

Next, portable terminal 300 searches for the menu offered by the selected food service establishment and, if the menu is disclosed, presents the menu to the user (S165). In step S166 after this, a food consumption log is input by portable terminal 300 receiving the user's selection of an eaten (or to be eaten) menu item. If the food service establishment also discloses nutritional information such as salt content in conjunction with the menu information, portable terminal 300 may obtain the nutritional information.

This makes it easy to identify the food service establishment where the meal was eaten and the menu of the meals served there, thereby reducing the time and effort required to input a food consumption log. At the same time, if nutritional information is disclosed, the accuracy of, for example, the amount of salt intake can be expected to improve.

It is known that meals from food service establishments tend to contain more salt than those prepared at home, even if the dish is the same. The reason is that a lot of salt tends to be used to make meals taste better and improve the preservation of food. For example, it is known that nikujaga consumed at food service establishments has a higher salt content than nikujaga prepared at home.

Therefore, if a meal is estimated to be from a food service establishment via the processes described above, the estimated salt intake corresponding to the input food consumption log may be calculated at a higher rate than normal (home-cooked meals). This makes it possible to take into account a higher-than-expected salt intake due to a meal being from a food service establishment, and inhibits deviations from the target salt intake.

### Second Example

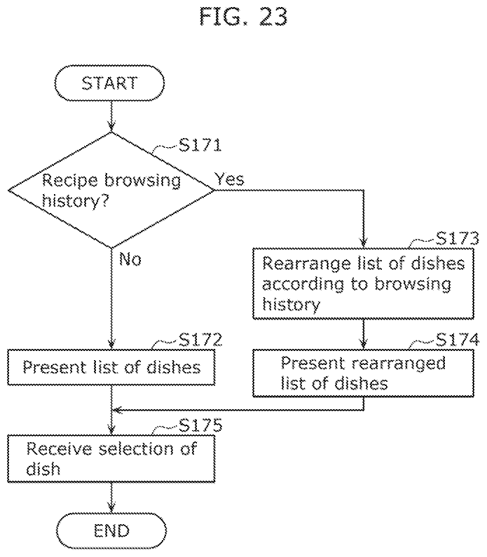

FIG. 23 is a flowchart illustrating a second example of a process of inputting a food consumption log.

The app that manages the food consumption logs on portable terminal 300 may have a function for searching for cooking recipes.

When the user uses the app on portable terminal 300 to input a food consumption log, portable terminal 300 determines whether there is a browsing history of cooking recipes (S171).

If portable terminal 300 determines that there is no browsing history of cooking recipes (No in S171), it presents the user with a list of dishes stored in the app or in server 100 connected via the internet (S172). In step S175 after this, a food consumption log is input by portable terminal 300 receiving the user's selection of an eaten (or to be eaten) dish. Other possible methods for inputting food consumption log entries include taking pictures.

If portable terminal 300 determines that there is a browsing history of cooking recipes (Yes in S171), it rearranges the list of dishes stored in the app or in server 100 connected via the internet according to the browsing history (S173). Specifically, portable terminal 300 rearranges the list of dishes so that dishes corresponding to the cooking recipes included in the browsing history are at the top of the list. When a cooking recipe is browsed, portable terminal 300 may include the dishes of the meal of the cooking recipe itself in the browsing history and present it to the user in the list of dishes.

Portable terminal 300 then presents the rearranged list of dishes to the user (S174). In step S175 after this, a food consumption log is input by portable terminal 300 receiving the user's selection of an eaten (or to be eaten) dish. Other possible methods for inputting food consumption log entries include taking pictures.

With this, dishes that are more likely to have been cooked will appear higher on the list, making it easier for users to select dishes they have eaten.

### Third Example

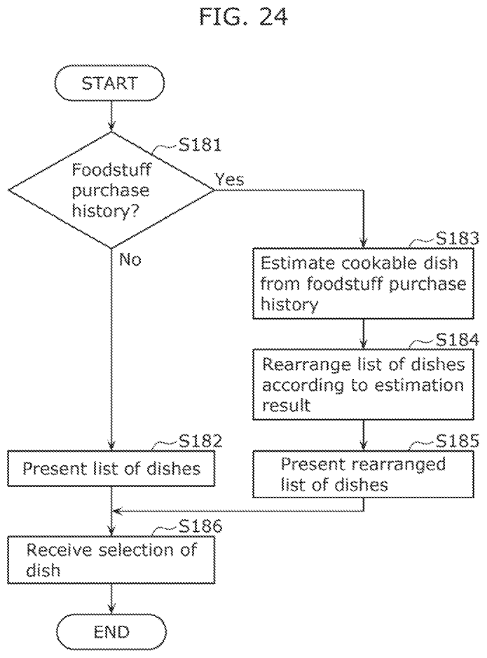

FIG. 24 is a flowchart illustrating a third example of a process of inputting a food consumption log.

The app that manages the food consumption logs on portable terminal 300 may have a function for storing or searching a purchase history of foodstuffs purchased from stores and the like.

When the user uses the app on portable terminal 300 to input a food consumption log, portable terminal 300 determines whether there is a purchase history of foodstuffs (S181).

If portable terminal 300 determines that there is no purchase history of foodstuffs (No in S181), it presents the user with a list of dishes stored in the app or in server 100 connected via the internet (S182). In step S186 after this, a food consumption log is input by portable terminal 300 receiving the user's selection of an eaten (or to be eaten) dish. Other possible methods for inputting food consumption log entries include taking pictures or extraction from the browsing history of cooking recipes.

If portable terminal 300 determines that there is a purchase history of foodstuffs (Yes in S181), it estimates a dish that could be cooked from the purchase history of foodstuffs (S183).

Next, portable terminal 300 rearranges the list of dishes stored in the app or in server 100 connected via the internet according to the result of the estimation (S184). Specifically, portable terminal 300 rearranges the list of dishes so that dishes corresponding to result of the estimation are at the top of the list.

Portable terminal 300 then presents the rearranged list of dishes to the user (S185). In step S186 after this, a food consumption log is input by portable terminal 300 receiving the user's selection of an eaten (or to be eaten) dish. Other possible methods for inputting food consumption log entries include taking pictures or extraction from the browsing history of cooking recipes.

With this, dishes that are more likely to have been cooked will appear higher on the list, making it easier for users to select dishes they have eaten.

### Fourth Example

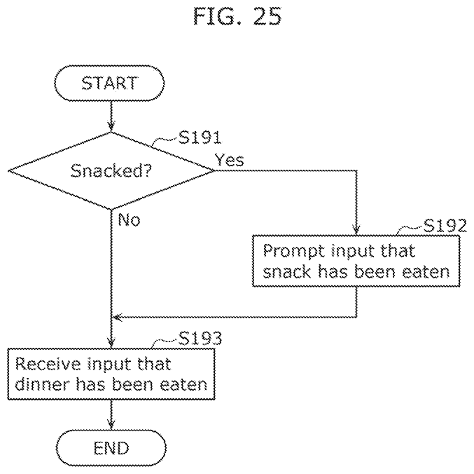

FIG. 25 is a flowchart illustrating a fourth example of a process of inputting a food consumption log.

When the user uses the app on portable terminal 300 to input a food consumption log, portable terminal 300 determines whether the user has eaten a snack (S191). More specifically, during the time of day at which dinner is typically eaten (for example, from 17:00 to 22:00), if no food consumption log has been input since the input for lunch, portable terminal 300 presents a UI that asks the user if they have eaten any snacks or other the like between lunch and dinner (i.e., if they have snacked between meals). Portable terminal 300 determines that the user has eaten a snack when an input indicating such is made, and determines that the user has not eaten a snack an input indicating such is made.

If portable terminal 300 determines that the user has eaten a snack (Yes in S191), it prompts the user to input that they have eaten a snack (S192).

If portable terminal 300 determines that the user has not eaten a snack (No in S191), or after step S192, it receives an input from the user that they have eaten dinner (S193).

This allows portable terminal 300 to receive an input that a snack was eaten along with an input that dinner has been eaten in cases in which the user ate a snack before dinner.

Portable terminal 300 may similarly confirm with the user that they did not eat a snack after dinner the previous night upon the user inputting a food consumption log for breakfast, and confirm with the user that they did not eat a snack after breakfast upon the user inputting a food consumption log for lunch.

Regarding snacks after dinner, portable terminal 300 may perform the detection before the user goes to bed and confirm with the user that they did not eat a snack after dinner.

Portable terminal 300 may confirm with the user that there are no omissions in entries in the food consumption log for the three meals (breakfast, lunch, and dinner) when checking before the user goes to bed.

When portable terminal 300 receives an input of a food consumption log for dinner from the user via the app that manages the food consumption logs, if the user has a history of meal delivery service usage in other apps, portable terminal 300 may obtain the history from that smartphone app and display it as an option for the input of the food consumption log.

The above acts as a prevention against forgetting to input food consumption log entries for snacks, which are easy to forget and can be a burden, and thus reduces the time and effort required for inputting food consumption log entries.

### Displaying Associated Blood Pressure Results and Food Consumption Log

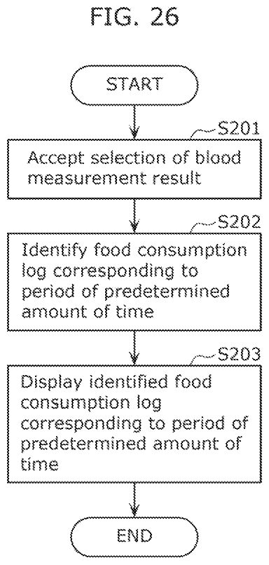

FIG. 26 is a flowchart illustrating one example of the process of displaying blood pressure measurement results and the food consumption log in association with each other.

Portable terminal 300 accepts the selection of one result among one or more blood pressure measurement results (S201).

Next, portable terminal 300 identifies, among the one or more first food consumption logs stored in storage 105, one or more second food consumption logs that are associated with a second timing included in period of a predetermined amount of time ending at a first timing at which the selected result was obtained (S202).

Next, portable terminal 300 displays the identified one or more second food consumption logs on display 304 (S203).

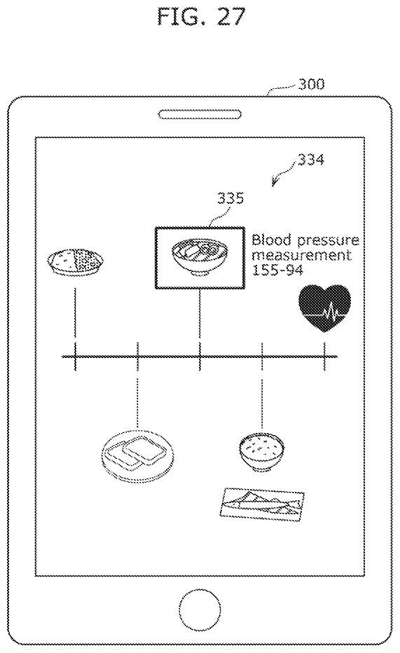

FIG. 27 illustrates one example of a UI including one or more second food consumption logs.

As illustrated in FIG. 27, UI 334 includes one selected blood pressure measurement result (maximum blood pressure 155 mmHg−minimum blood pressure 94 mmHg) as well as the food consumption log (second food consumption log) for a period of a predetermined amount of time (for example, 24 hours) before the result.

For example, if the one blood pressure measurement result (maximum blood pressure 155 mmHg−minimum blood pressure 94 mmHg) was measured before bedtime, the food consumption log for 24 hours before the timing of the measurement taken before bedtime may be displayed. The food consumption log includes dinner (rice and grilled fish), lunch (ramen noodles), breakfast (toast), and dinner from the previous day (curry and rice).

Among the displayed food consumption logs, those with the greatest impact on blood pressure may be emphasized. For example, in FIG. 27, food consumption log 335 corresponding to ramen noodles is emphasized by being framed. Examples of emphasis other than framing include displaying the food consumption log to be emphasized larger than others. Displayed food consumption logs that have an adverse effect on blood pressure may be emphasized with a color that indicates danger (for example, red, yellow, etc.) and consumed food that has a positive effect on blood pressure may be emphasized with a color that indicates safety (for example, blue, green, etc.).

If there are more than one displayed food consumption logs that have a significant effect on blood pressure, one or more closer to the blood pressure measurement time (origin point) may be emphasized.

Effect on blood pressure may be determined by the amount of salt in the food or by the amount of energy in the food. Stated differently, the effect on blood pressure may be determined to increase as the amount of salt increases. Similarly, the effect on blood pressure may be determined to increase as the amount of energy increases.

Emphasis may be placed on one or more third food consumption logs that exceed a predetermined amount of salt among one or more second food consumption logs. Emphasis may be placed on the most recent third food consumption log among one or more third food consumption logs that exceed a predetermined amount of salt among one or more second food consumption logs. Emphasis may be placed on one or more third food consumption logs that exceed a predetermined amount of energy among one or more second food consumption logs. Emphasis may be placed on the most recent third food consumption log among one or more third food consumption logs that exceed a predetermined amount of energy among one or more second food consumption logs.

### Advantageous Effects, etc.

Lifestyle improvement system 10 according to the present embodiment includes obtainer 101, determiner 102, and notifier 103. Obtainer 101 obtains wake-up information about an act of waking up by the user and breakfast preparation information about an act of preparing breakfast by the user. Determiner 102 determines an optimal timing for the user's blood pressure measurement based on the wake-up information and the breakfast preparation information. Notifier 103 notifies timing information regarding the timing as determined by determiner 102.

With this, the lifestyle improvement system can support the user to take blood pressure measurement at appropriate timings since it notifies the user with timing information regarding the optimal timing for the user to measure their blood pressure, which is determined based on the wake-up information and breakfast preparation information.

In lifestyle improvement system 10, the timing is included in a period from after the user wakes up to when the user starts the act of preparing breakfast. This can support blood pressure measurement during a period of low blood pressure variability.

In lifestyle improvement system 10, obtainer 101 further obtains urination information about urination by the user. Determiner 102 further determines the timing based on the urination information.

With this, the lifestyle improvement system can support the user to take blood pressure measurement at more appropriate timings since it notifies the user with timing information regarding the optimal timing for the user to measure their blood pressure, which is determined based on the wake-up information, urination information, and breakfast preparation information.

In lifestyle improvement system 10, the timing is included in a period from after the user urinates to when the user starts the act of preparing breakfast. This can support blood pressure measurement during a period of even lower blood pressure variability.

Lifestyle improvement system 10 further includes an informer. Based on the timing information, the informer informs the user so as to prompt the user to measure their blood pressure.

With this, the lifestyle improvement system can support the user to take blood pressure measurement at appropriate timings since it can prompt the user to take a blood pressure measurement at the optimal timing for the user to measure their blood pressure.

Lifestyle improvement system 10 further includes measurement device 204 as the measurer. Measurement device 204 measures the user's blood pressure based on the timing information. This allows the user to take a blood pressure measurement at the optimal timing for the user to measure their blood pressure.

Lifestyle improvement system 10 further includes storage 105 and extractor 104. Storage 105 stores each of one or more results of the blood pressure measurement of the user in association with a time of obtainment of the result. Based on the timing information, extractor 104 extracts, from among one or more results, the result of a blood pressure measurement taken at the optimal timing for blood pressure measurement by the user.

With this, the lifestyle improvement system can identify the result of a blood pressure measurement taken at the appropriate timing since it can identify the optimal timing for the user to measure their blood pressure.

In lifestyle improvement system 10, obtainer 101 further obtains bed-time information about an act of going to bed by the user and history information indicating a history of blood pressure measurement by the user. Determiner 102 further determines whether the user has measured their blood pressure in a period of a predetermined amount of time ending when the user goes to bed, based on the bed-time information and the history information. Notifier 103 notifies the timing information when determiner 102 determines that the user has not taken a blood pressure measurement in the period.

With this, the lifestyle improvement system can support the user to take blood pressure measurement at an appropriate timing before bed since it notifies the user with timing information when the user has not taken their blood pressure measurement at the optimal timing before going to bed for the user to measure their blood pressure, based on the bed-time information and the history information.

Lifestyle improvement system 10 according to the present embodiment includes obtainer 101, determiner 102, and notifier 103. Obtainer 101 obtains bed-time information about an act of going to bed by the user and history information indicating a history of blood pressure measurement by the user. Determiner 102 determines whether the user has measured their blood pressure in a period of a predetermined amount of time ending when the user goes to bed, based on the bed-time information and the history information. Notifier 103 notifies the timing information regarding the optimal timing for the user to measure their blood pressure when determiner 102 determines that the user has not taken a blood pressure measurement in the period.

With this, the lifestyle improvement system can support the user to take blood pressure measurement at an appropriate timing before bed since it notifies the user with timing information when the user has not taken their blood pressure measurement at the optimal timing before going to bed, based on the bed-time information and the history information.

Lifestyle improvement system 10 further includes storage 105. When determiner 102 determines that blood pressure has been measured in the period of a predetermined amount of time ending when the user goes to bed, storage 105 stores a result of a blood pressure measurement taken in that period as a result obtained at the optimal timing for blood pressure measurement.

With this, the lifestyle improvement system can identify the result of a blood pressure measurement taken at an appropriate timing before going to bed since it can identify the optimal timing for the user to measure their blood pressure before going to bed.

Lifestyle improvement system 10 includes storage 105, display 304, input receiver 302, and controller 303. Storage 105 stores one or more results of the blood pressure measurement of the user each in association with a first timing at which the result was obtained, and stores one or more first food consumption logs each in association with a second timing at which the user consumed the food and/or drink indicated in the first food consumption log. Input receiver 302 accepts a selection of one result among one or more results. Controller 303 identifies, among the one or more first food consumption logs, one or more second food consumption logs that are associated with a second timing included in period of a predetermined amount of time ending at a first timing at which one result was obtained, and displays the identified one or more second food consumption logs on display 304.

With this, since one or more second food consumption logs that are associated with the second timing included in period of a predetermined amount of time ending at the first timing at which one blood pressure measurement result was obtained are displayed on the display, the user can easily recognize one or more second food consumption logs that may have influenced the one result.

In lifestyle improvement system 10, each of the one or more first food consumption logs includes an amount of salt included in a food and/or drink indicated by the first food consumption log. Controller 303 further emphasizes and displays one or more third food consumption logs that exceed a predetermined amount of salt among one or more second food consumption logs.

With this, from among one or more second food consumption logs, the user can easily recognize one or more third food consumption logs that may have influenced the one blood pressure measurement result.

In lifestyle improvement system 10, each of the one or more first food consumption logs includes an amount of salt included in a food and/or drink indicated by the first food consumption log. Controller 303 further emphasizes and displays a most recent third food consumption log among one or more third food consumption logs that exceed a predetermined amount of salt among one or more second food consumption logs.

With this, from among one or more second food consumption logs, the user can easily recognize the most recent third food consumption log that exceeds a predetermined amount of salt.

In lifestyle improvement system 10, each of the one or more first food consumption logs includes an amount of energy included in a food and/or drink indicated by the first food consumption log. Controller 303 further emphasizes and displays one or more third food consumption logs that exceed a predetermined amount of energy among one or more second food consumption logs.

With this, from among one or more second food consumption logs, the user can easily recognize a third food consumption log that exceeds a predetermined amount of energy.

In lifestyle improvement system 10, each of the one or more first food consumption logs includes an amount of energy included in a food and/or drink indicated by the first food consumption log. Controller 303 further emphasizes and displays a most recent third food consumption log among one or more third food consumption logs that exceed a predetermined amount of energy among one or more second food consumption logs.

With this, from among one or more second food consumption logs, the user can easily recognize the most recent third food consumption log that exceeds a predetermined amount of energy.

### Variations

In lifestyle improvement system 10 according to the above embodiment, portable terminal 300 may include all or part of the functions of obtainer 101, determiner 102, notifier 103, extractor 104, and storage 105 included in server 100. In lifestyle improvement system 10, server 100 may include all or part of the functions of controller 303 included in portable terminal 300. If portable terminal 300 includes all or part of the functions of obtainer 101, determiner 102, notifier 103, extractor 104, and storage 105 included in server 100, devices 201 through 203 may transmit the sleep information, the urination information, and the breakfast preparation information to portable terminal 300. Portable terminal 300 may notify measurement device 204 with the timing information.

### Other Comments

Each of the elements in each of the above-described embodiments may be implemented as dedicated hardware, or may be realized by executing a software program suitable for the element. Each of the elements may be realized by way of a program executing unit, such as a CPU or a processor, reading and executing the software program recorded on a recording medium such as a hard disk or a semiconductor memory.

As described above, embodiments have been described as examples of techniques according to the present disclosure. The accompanying drawings and detailed description are provided for this purpose.

Therefore, elements described in the accompanying drawings and the detailed description include, in addition to elements essential to overcoming the technical problem, elements that are not essential to overcoming the technical problem but are included in order to exemplify the techniques described above. As such, description of these non-essential elements in the accompanying drawings and the detailed description should not be taken to mean that these non-essential elements are essential.

The above embodiments are for providing examples of the techniques according to the present disclosure, and thus various modifications, substitutions, additions, and omissions are possible within the scope of the claims and equivalents thereof.

### Industrial Applicability

The present disclosure is applicable to lifestyle improvement systems that can support taking blood pressure measurements at appropriate timings.

1-8. (canceled)

9. A lifestyle improvement system comprising:
an obtainer that obtains bed-time information about an act of going to bed by a user and history information indicating a history of blood pressure measurement by the user;
a determiner that determines whether the user has taken a blood pressure measurement in a period of a predetermined amount of time ending when the user goes to bed, based on the bed-time information and the history information; and
a notifier that notifies the user with first timing information regarding an optimal first timing for blood pressure measurement when the determiner determines that the user has not taken the blood pressure measurement in the period.

10. The lifestyle improvement system according to claim 9, further comprising:
an informer that prompts the user to measure blood pressure, based on the first timing information.

11. The lifestyle improvement system according to claim 9, further comprising:
a measurer that measures a blood pressure of the user, based on the first timing information.

12. The lifestyle improvement system according to claim 9, further comprising:
storage that, when the determiner determines that the user has taken the blood pressure measurement in the period, stores a result of the blood pressure measurement taken in the period as a result obtained at the optimal timing for blood pressure measurement.

13. (canceled)

14. A portable terminal connected to a server over a network,
the server including:
an obtainer that obtains bed-time information about an act of going to bed by a user and history information indicating a history of blood pressure measurement by the user;
a determiner that determines whether the user has taken a blood pressure measurement in a period of a predetermined amount of time ending when the user goes to bed, based on the bed-time information and the history information; and
a notifier that notifies the user with timing information regarding an optimal timing for blood pressure measurement when the determiner determines that the user has not taken the blood pressure measurement in the period,
the portable terminal comprising:
a receiver that receives the timing information; and
an informer that informs the user of an optimal timing to measure blood pressure, based on the timing information.

15-16. (canceled)

17. A control method of a lifestyle improvement system including one or more processors and an informer, the control method comprising:
obtaining, by the one or more processors, bed-time information about an act of going to bed by a user and history information indicating a history of blood pressure measurement by the user; and
informing, by the informer, the user of timing information regarding an optimal timing for blood pressure measurement when it is determined, based on the bed-time information and the history information, that the user has not taken a blood pressure measurement in a period of a predetermined amount of time ending when the user goes to bed.

18. A non-transitory computer-readable recording medium having recorded thereon a program for causing a computer to execute the control method according to claim 17.

19. The lifestyle improvement system according to claim 9, wherein
the obtainer further obtains wake-up information about an act of waking up by the user and breakfast preparation information about an act of preparing breakfast by the user,
the determiner further determines an optimal second timing for blood pressure measurement of the user based on the wake-up information and the breakfast preparation information, and
the notifier further notifies second timing information regarding the optimal second timing determined by the determiner.

20. The lifestyle improvement system according to claim 19, wherein
the optimal second timing is included in a period from after the user wakes up to when the user starts the act of preparing breakfast.

21. The lifestyle improvement system according to claim 19, wherein
the obtainer further obtains urination information about urination by the user, and
the determiner further determines the optimal second timing based on the urination information.

22. The lifestyle improvement system according to claim 19, wherein
the optimal second timing is included in a period from after the user urinates to when the user starts the act of preparing breakfast.

23. The lifestyle improvement system according to claim 19, further comprising:
an informer that prompts the user to measure blood pressure, based on the second timing information.

24. The lifestyle improvement system according to claim 19, further comprising:
a measurer that measures a blood pressure of the user, based on the second timing information.

25. The lifestyle improvement system according to claim 19, further comprising:
storage that stores each of one or more results of the blood pressure measurement of the user in association with a time of obtainment of the result; and
an extractor that extracts, from the one or more results, a result of a blood pressure measurement taken at the optimal second timing for blood pressure measurement of the user, based on the second timing information.
